(19) United States
(12) Patent Application Publication
(10) Pub. No.: US 2003/0054411 A1
(43) Pub. Date: Mar. 20, 2003

(54) GPR motifs and uses thereof

(76) Inventor: Stephen M. Lanier, New Orleans, LA (US)

Correspondence Address: Gwendolyn D. Spratt, Esq., NEEDLE & ROSENBERG, P.C., The Candler Building, 127 Peachtree Street, N.E., Atlanta, GA, 30303-1811, US

(21) Appl. No.: 10/234,668

(22) Filed: Sep. 3, 2002

Related U.S. Application Data
(60) Provisional application No. 60/316,556, filed on Aug. 31, 2001.

(51) Int. Cl. G01N 33/53; C07H 21/04; C07K 14/705; C07K 14/435; C12P 21/02; C12N 5/06

(52) U.S. Cl. 435/7.1; 435/69.1; 435/320.1; 435/325; 530/350; 536/23.5

(57) ABSTRACT

The present invention provides a polypeptide of less than 600 amino acids comprising the sequence set forth as SEQ ID NO:1. SEQ ID NO: 1 is an example of a GPR motif. The invention also provides a polypeptide consisting of the sequence set forth as SEQ ID NO:1. Also provided is a method of inhibiting G protein mediated signal transduction comprising administering to a subject a GPR motif or mimetic of a GPR motif.

This application claims the benefit of U.S. application Ser. No. 60/316,556, filed Aug. 31, 2001 which is hereby incorporated by reference in its entirety. This application also claims the benefit of U.S. application Ser. No. 09/746,341, filed Dec. 21, 2000 which claims priority to U.S. application Ser. No. 60/171,726, filed Dec. 22, 1999 and which are both hereby incorporated by reference in their entirety. This application also claims the benefit of U.S. application Ser. No. 09/307,105 filed May 7, 1999, which claims priority to U.S. application Ser. No. 60/084,841 filed May 8, 1998, and which applications are hereby incorporated herein by reference in their entirety.

### SUMMARY OF THE INVENTION

The present invention provides a polypeptide of less than 600 amino acids comprising the sequence set forth as SEQ ID NO:1.

The invention further provides a method of identifying a polypeptide as a putative receptor-independent activator of heterotrimeric G-protein signaling pathways comprising detecting SEQ ID NO:1 in a polypeptide, wherein the presence of SEQ ID NO:1 in the polypeptide indicates the polypeptide is a putative receptor-independent activator of heterotrimeric G-protein signaling pathways.

Also provided by the present invention is a method of screening for a mimetic or analog of SEQ ID NO: 1 or a fragment thereof, comprising: a) contacting a model system known to express an activity of SEQ ID NO: 1 with a putative mimetic or analog; b) detecting the presence or absence of the known activity of SEQ ID NO: 1 in the presence of the putative mimetic or analog; and c) correlating the presence or absence of the activity with the presence of a mimetic of SEQ ID NO: 1.

Further provided by the present invention is a method of stimulating G-protein mediated signal transduction in a subject comprising administering to a subject a GPR motif or a mimetic of a GPR motif.

The invention also provides a method of screening for an antagonist of SEQ ID NO: 1 is provided, comprising: a) contacting a model system known to express an activity of a polypeptide comprising a GPR motif with a putative antagonist in the presence of SEQ ID NO: 1 or a fragment thereof; b) detecting the absence of, or reduction in, the known activity of the polypeptide comprising a GPR motif, in the presence of the putative antagonist; c) correlating the absence of, or reduction in, the activity with the presence of an antagonist of SEQ ID NO: 1 or a fragment thereof and; d) confirming that the antagonist interacts with SEQ ID NO: 1 or interferes with the binding of SEQ ID NO: 1 to a G-protein.

### DESCRIPTION OF THE FIGURES

FIG. 1 shows the effect of partially purified NG10815 G-protein activator on GTPγ³⁵S to heterotrimeric purified bovine brain G-protein and transducin. (Sato M, Ribas C, Hildebrandt J D and Lanier S M. Characterization of a G-protein activator in the neuroblastoma glioma cell hybrid NG108-15. J Biol Chem (1996) 271:30052-30060). Heterotrimeric G-proteins were preincubated with or without the partially purified material from NG10815 cells for 1 hr at 4° C. Preincubation cocktail=5 μM GDP, 6.25 nM brain G-protein or 50 nM transducin. 15 μl NG108-15 activator in a total volume of 25 ul with final buffer concentrations of 0.066% Thesit, 50 mM Tris-HCl Ph.7.4, 5 mM MgCl₂, 0.6 mM EDTA. After this preincubation period, 10 μl of this cocktail for each sample was added to 40 μl assay mixture containing GTPγ³⁵S. The final buffer composition in the assay mixture tube was 50 mM Tris-HCl pH 7.4, 0.6 mM EDTA, 5 mM MgCl₂, 150 mM NaCl, 1 mM DTT, ˜230,000 cpm (˜2 nM) GTPγ³⁵S, 1 uM GDP and 0.013% Thesit. Incubation was then continued for different times at 24° C. and terminated by vacuum filtration on BA85 (S & S) nitrocellulose membranes. The data are presented as the average of two experiments with duplicate determinations. GTPγ³⁵S binding to the NG108-15 cell extract alone was determined for each time point and subtracted from the corresponding sample preincubated with G-protein to generate the numbers in the curves corresponding to G-protein preincubated partially purified NG10815 extract. Using this assay system with partially purified NG10815 extract, the actual dpms bound at 30 minute incubation time points for experiments with brain G-protein are: G-protein alone—˜2000, NG10815 extract—˜3,000 to 5,000, G-protein preincubated with NG10815 extract—˜56,000. Partially purified NG10815 extract refers to material processed as described in general methods. NOTE: This action of the partially purified material was still observed with brain G-protein that had been ADP-ribosylated with pertussis toxin. The partially purified NG10815 extract also increased GTPγ³⁵S to membrane bound G-proteins in similar assays using a preparation of DDT1-MF2 cell membranes. The action of the partially purified NG10815 extract in membrane preparations was also pertussis toxin-insensitive. α2-adrenergic receptor mediated increases in GTPγ³⁵S binding in the same membrane preparations were completely blocked by pertussis toxin pretreatment of the cells.

FIG. 2 shows the time course for GTPγ³⁵S binding to Gα in the presence and absence of the NG10815 G-protein activator. G-protein a subunits were preincubated with or without the partially purified material from NG10815 cells for 1 hr at 4° C. Preincubation cocktail=5 μM GDP, 50 nM Gα, 15 μl NG108-15 activator in a total volume of 25 ul with final buffer concentrations of 0.066% Thesit, 50 mM Tris-HCl Ph.7.4, 5 mM MgCl₂, 0.6 mM EDTA. After this preincubation period, samples were processed and assayed as described in the legend to FIG. 1. The data are presented as the average of two experiments with duplicate determinations. GTPγ³⁵S binding to the NG108-15 cell extract alone was determined for each time point and subtracted from the corresponding sample preincubated with G-protein to generate the numbers in the curves corresponding to G-protein preincubated with partially purified NG10815 extract. GTPγ³⁵S bound at 30 minutes (dpm): Goa (G alone˜9000, G+extract˜103,000), Gα i1 (G alone˜2500, G+extract˜14,500),Gα i2 (G alone˜2000, G+extract˜8000), Gα i3 (G alone˜1400, G+extract˜8000, extract alone˜6,000).

FIG. 3 shows the effect of increasing amounts of the NG10815 G-protein activator on GTPγ³⁵S binding to Ga subunits. G-protein a subunits were preincubated with or without increasing amounts of partially purified material from NG10815 cells for 1 hr at 4° C. Preincubation cocktail=5 μM GDP, 50 nM Gα, 15 μl NG108-15 activator in a total volume of 25 ul with final buffer concentrations of 0.066% Thesit, 50 mM Tris-HCl Ph.7.4, 5 mM MgCl₂, 0.6 mM EDTA. After this preincubation period, samples were processed and assayed as described in the legend to FIG. 1 and samples incubated for 30 minutes at 24° C. and terminated by vacuum filtration on BA85 (S & S) nitrocellulose membranes. Data are presented as the average of two experiments with duplicate determinations. GTPγ³⁵S binding to the NG108-15 cell extract alone was determined for each concentration of cell extract and subtracted from the corresponding sample preincubated with G-protein to generate the values presented.

FIG. 4 shows the influence of NG10815 G-protein activator on GDP dissociation from Gα. 20 nM Goα was incubated in the presence or absence (vehicle) of 10 μl NG108-15 activator in buffer containing with 10 μg/ml BSA, 2.3 μl GTPα³²P, 1 μM GTP, 50 mM Tris-HCl pH 7.6, 5 mM EDTA, 1 mMDTT, 0.1% Triton X-100, 10 mM MgCl₂ in a total volume of 200 μl with 0.005% Thesit at 30° C. for 20minutes. Samples were then cooled to 24° C. and dissociation initiated by addition of 1 mM GDP. 10 μl aliquots of the 200 ul for each sample (plus or minus NG10815 extract) were processed by vacuum filtration just prior to addition of GDP and at increasing times following the addition of GDP. The data shown are representative of three experiments. Goα was expressed in and purified from Sf9 cells and provided by Stephen Graber (Department of Pharmacology, West Virginia University School of Medicine). Dpm bound at time zero prior to addition of GDP were 10,000 to 12,000 and these values were taken as 100% bound.

FIG. 5 illustrates a protein identified by searching GenBank with the GPR consensus motif.

FIG. 6 shows the activation of the pheromone response pathway by NG108-15 cDNA clones. (A) Three cDNA clones (#34, #37 and #53) were isolated from a screen of 1.1×10⁶ yeast transformants based upon their ability to promote growth in a galactose-dependent manner in the yeast expression cloning system (5). B) Analysis of the cDNA clones in spot growth assays using yeast strains lacking STE20, STE5 or STE4. Control (Non-induced/no selection)—glucose media lacking uracil and tryptophan (UT). Non-induced/selection—glucose media lacking uracil/tryptophan/histidine (UTH) and containing the histidine analog aminotriazole (AT) at 1 mM. Induced/selection—galactose media lacking UTH and containing 1 mM AT. WT—wild type strain CY1316/1183. cDNA #34 was active in the absence of a functional STE5 and thus was not evaluated (black circles in key) in yeast strains lacking STE4 or STE20. For spot growth assays, ˜2,000 cells suspended in water were spotted on appropriate medium and the plates were incubated at 30° for 2 days prior to photography. Experiments in (A, B) were repeated three times with similar results.

FIG. 7 shows the functional and biochemical properties of AGS2 and AGS3. (A) Bioactivity of cDNA #37 and 53 in yeast strains expressing different Gα subunits. The Gpa1, Giα2_{Gpa1(1-41)}, Giα3_{Gpa1(1-41)} and Gα16_{Gpa1(1-41)} yeast strains expressed similar amounts of G-protein as determined by immunoblotting with Gpa1-specific antisera. Each series of experiments include data obtained in the absence of Gα to indicate the signal obtained when the action of Gβγ is not limited by reformation of heterotrimer. Data are presented as the mean±S.E.M of three experiments with duplicate determinations. (B) Interaction of AGS2 and AGS3 with G-proteins. AGS2 and the bioactive AGS3 peptide (74 amino acids) beginning at the first in frame methionine were generated as GST-fusion proteins in bacteria and purified using a glutathione affinity matrix. AGS2 and AGS3 GST fusion proteins were incubated with G-protein subunits (Gα=60-80 nM, Gβγ=40 nM, fusion protein=˜300 nM, total volume=250 ul) for 12 hrs at 4° C. GDP=10 uM. GTPγS=10 uM plus 5 mM MgCl₂. Proteins were then adsorbed to glutathione matrix and retained G-protein subunits identified by immunoblotting following gel electrophoresis. Giα2 was generated in Sf9 cells as an amino terminal his6-tagged protein and was detected on membrane transfers using anti-Xpress antisera (Invitrogen). Bovine brain Gβγ was detected on membrane transfers with Gβ antisera. Input refers to 20 ul of the incubation mixture. The results presented are representative of four separate experiments. The GST-AGS3 fusion protein was functionally similar to the original AGS3 isolate in terms of its ability to promote growth. In contrast, the GST-AGS2 fusion protein did not promote growth in the yeast assay system suggesting that the amino terminus region of AGS2 has an important functional role. C) Effect of G204A Giα2 substitution and RGS4 on the functionality of AGS proteins. The G204A Giα2 yeast strain and the system used for over expression of RGS4 are described in detail in reference 5. Protein expression was confirmed by immunoblotting. AGS 1 was discovered in a similar screen using a human liver cDNA library (5, 7). WT—wild type strain CY1316/1183 containing Giα2 as described for the original yeast screen. RGS4 was overexpressed in the CY1316/1183 yeast strain. Similar results were obtained in three experiments.

FIG. 8 shows the consensus sequence for G-protein regulators. A) Sequence of full length rat AGS3. The shaded and overlined sequences represent the tetratricopeptide repeat motif and a repeated segment of amino acids (I-IV), respectively, as discussed in the text. The underlined amino acids indicate the fragment (cDNA #53) isolated in the original yeast screen. B) The four repeat domains in the carboxyl terminus half of AGS3, human LGN, the predicted C. elegans protein and related sequences identified by the motif search program MEME were aligned and used to define a consensus sequence as described in the Examples. Right panel—Interaction of wild type AGS3 and AGS3 mutants with Giα2 in mammalian cell extracts. The AGS3 eDNA isolated in the yeast screen and shown to interact with Gα subunits (FIG. 2B) was modified by site-directed mutagenesis to disrupt conserved residues within the GPR motif. The number of the amino acids altered in the mutant constructs (F8R, Q15A and R23F) refers to their position in the sequence found in the left panel aligning the conserved domains. The GST-AGS3 fusion proteins were added to DDT1-MF2 cell lysates containing 30 uM GDP and processed to determine interaction with endogenous mammalian Giα2 or Giα3 as described in the Examples. Equal loading of individual samples was verified by amido black staining or immunoblotting with AGS3 specific antisera. The lysate lane contains 10 ul of the 250 ul sample incubated with the fusion proteins. C) 225 ul of lysate containing nucleotide (30 uM) were incubated with GST or GST-AGS3 (300 nM) for 30 minutes at 24° C. Protein complexes were isolated by glutathione affinity matrix or immunoprecipitation with Giα3 antisera and processed as described in Examples. The input refers to 10 ul of the cell lysate. Giα subunits were detected on membrane transfers with the Giα3 antibody and Gβ was detected with an amino terminus antibody. The images in (B) and (C) are representative of two to four experiments using different batches of -fusion protein and cell extracts.

FIG. 9 shows the alignment of the GPR motifs found in AGS3 and related proteins. The overall domain structure of AGS3 (650-amino acid protein) is indicated at the top of the figure. The hashed boxes represent the TPR domain. The GPR domains of rat AGS3 (AAF08683), human AGS3 (CAB5595 1), the D. melanogaster PINS protein (AAF36967), the Caenorhabditis elegans protein (CE) (AAA81387), and the Tetraodon nigroviridis (puffer fish) protein (AL338846) were aligned by PILEUP (Genetics Computer Group Wisconsin Package) and visual adjustment. A consensus amino acid was defined by the presence of an amino acid or closely related residue in all four GPR repeats.

FIG. 10 shows the influence of GPR peptides on the interaction of GST-AGS3-GPR with Giα and GTPγS binding to Giγ. A and B, the carboxyl region of AGS3 (Pro⁴⁶³-Ser⁶⁵⁰) containing all four GPR repeats was generated as a His-tagged (A) or GST fusion (B) protein for protein interaction assays as described under Examples. All interactions were done in the presence of 10 μM GDP, and the input lanes represent one-tenth of the G-protein used in each interaction assay. A, Giα1 (75 nM) was incubated with 300 nM His-tagged AGS3-GPR in the absence and presence of increasing amounts of the GPR peptide, after which bound Giα was isolated on a nickel affinity matrix, and samples were processed for immunoblotting with Giα antisera. The blot in the upper panel of A was stripped and reprobed with AGS3 antisera to provide internal controls for protein loading. B, Giα2 (75 nM) was incubated with 300 nM AGS3-GPR or GST in the presence and absence of 100 μM GPR consensus peptide, GPR peptide Q15A, or GPR peptide R23F. The immunoblots presented in A and B are representative of three experiments. C and D, GTPγ³⁵S binding to G-proteins (100 nM) was measured in the absence and presence of peptides as described in the Examples. Data are expressed as the percent of specific binding (˜5 pmol) observed in the absence of peptide and represent the means±S.E. derived from three experiments. The concentration of peptides in D is 10 μM.

FIG. 11 illustrates the stabilization of the GDP-bound conformation of Giα2 by the GPR consensus peptide. A, ³H-GDP dissociation from Giα2 (100 nM). Giα2 was loaded with ³H-GDP, and dissociation was initiated by addition of GTPΔS (100 μM) as described in the Examples. Data are expressed as the percent of specific binding observed in control samples for each time point that did not receive 100 μM GTPγS. The peptide concentration was 10 μM. B, Giα2 (100 nM) was loaded with ³H-GDP and incubated with 300 nM GST or GST-AGS3-GPR in the presence and absence of 10 μM GPR consensus peptide and processed for protein interactions as described in the Examples. The proteins bound to the glutathione affinity matrix were eluted, and the amount of bound GDP was measured by liquid scintillation spectroscopy. Data in A and B are presented as the means±S.E. derived from three experiments.

FIG. 12 shows the influence of GPR peptides on receptor interaction with G-proteins. Sf9 cell membranes expressing 5-HT_{1A} receptors were reconstituted with G-proteins in the presence and absence of GPR peptides as described in the Examples. The final concentration of peptide was 114 μM. Radioligand binding assays used a concentration of ³H-5HT near the K_{d} for the high affinity, guanosine 5′-(β,γ-imido)triphosphate-sensitive binding site (38, 40). The control bar indicates the amount of agonist binding observed in the absence of added G-protein. Data are presented as the mean±S.E. from four independent experiments.

### DETAILED DESCRIPTION OF THE INVENTION

The present invention provides an isolated polypeptide consisting of the sequence set forth in the sequence listing as SEQ ID NO:1. SEQ ID NO: 1 is an example of a G-protein regulatory (GPR) consensus sequence or motif that has the sequence [EDSQ][EN][X₁ or DGHVC][FLV][FVLMI][DESNA][LMI][LIV]X [RKNSH]XQ[SGA]X[RG][AIML][DENH][DE]QR (SEQ ID NO: 1) wherein X indicates any amino acid residue or no amino acid residue, [EDSQ] indicates any one of E, D, S or Q, [EN] indicates any one of E or N, [X₁ or DGHVC] indicates no amino acid residue or any one of D, G, H, V or C, [FLV] indicates any one of F, L or V, [FLVMI] indicates any one of F, L, V, M or I, [DESNA] indicates any one of D, E, S, N or A, [LMI] indicates any one of L, M or I, [LIV] indicates any one of L, I or V, [RKNSH] indicates any one of R, K, N, S or H, [SGA] indicates any one of S, G or A, [RG] indicates any one of R or G, [AIML] indicates any one of A, I, M or L, [DENH] indicates any one of D, E, N or H, and [DE] indicates any one of D or E.

The motif can be characterized by an invariant Q downstream of hydrophobic amino acid residues adjacent to acidic residues. An invariant Q can be present upstream of acidic residues adjacent to another invariant Q.

This invention also provides a polypeptide comprising SEQ ID NO: 1 that is shorter than the full length polypeptide in which it is found in nature, e.g. a polypeptide that is less than 600 amino acids in length and fragments thereof, wherein the polypeptide is not AGS3. AGS3 is defined in U.S. Ser. No. 09/307,105. This invention also provides polypeptides that comprise SEQ ID NO: 1 that are less than 500, 400, 300, 200, 100, 90, 80, 70, 60, 50, 45, 40, 35, 30, 25, 20, 15 and 10 amino acids in length or any length in between. The invention also provides fragments of unmodified and modified GPR motifs. The polypeptide fragments of the present invention can be recombinant proteins obtained by cloning nucleic acids encoding the polypeptide in an expression system capable of producing the polypeptide fragments thereof. For example, one skilled in the art can determine the regions of a GPR consensus sequence which can interact with another protein and cause a biological effect associated with the GPR consensus sequence. In one example, amino acids found to not contribute to either the activity, binding specificity, or other biological effect associated with the GPR consensus sequence can be deleted and/or substituted without a loss in the respective activity. The fragments, whether attached to other sequences or not, can also include insertions, deletions, substitutions, or other selected modifications of particular regions or specific amino acid residues, provided the activity of the peptide is not significantly altered or impaired. Further contemplated are polypeptides encoded by fragments of the nucleic acids provided herein. It is noted that the GPR consensus sequence of motif shown in SEQ ID NO: 1 is a specific example of a GPR consensus sequence, and that other examples of a GPR motif having slightly different sequences may be found in nature using routine protocols or generated by design based on the disclosure herein of SEQ ID NO: 1. One of skill in the art will recognize amino acid sequences having all or a majority of the amino acids of the consensus sequence are defined herein as potentially having a specified GPR motif. For example, an amino acid sequence having all but one amino acid residue of a consensus sequence may be recognized by one of skill in the art as potentially having a GPR motif. Likewise, an amino acid sequence having all but two amino acid residues of a consensus sequence may be recognized by one of skill in the art as potentially having such a GPR motif. Any polypeptide that potentially has a GPR motif can be confirmed to have or not have such a motif using routine techniques in a routine amount of experimentation. Examples of such confirmatory techniques are provided herein.

These polypeptides of this invention can also be obtained in any of a number of procedures well known in the art. One method of producing a polypeptide is to link two peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to a particular protein can be synthesized by standard chemical reactions. For example, a peptide or polypeptide can be synthesized and not cleaved from its synthesis resin whereas the other fragment of a hybrid peptide can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form a larger polypeptide. (Grant, Synthetic Peptides: A User Guide, W. H. Freeman and Co., N.Y. (1992) and Bodansky and Trost, Ed., Principles of Peptide Synthesis, Springer-Verlag Inc., N.Y. (1993)). Alternatively, the peptide or polypeptide can be independently synthesized in vivo as described above. Once isolated, these independent peptides or polypeptides may be linked to form a larger protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al. Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation, Science, 266:776-779 (1994)). The first step is the chemoselective reaction of an unprotected synthetic peptide-%-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Clark-Lewis et al. FEBS Lett., 307:97 (1987), Clark-Lewis et al., J.Biol.Chem., 269:16075 (1994), Clark-Lewis et al. Biochemistry, 30:3128 (1991), and Rajarathnam et al. Biochemistry, 29:1689 (1994)).

Alternatively, unprotected peptide segments can be chemically linked where the bond formed between the peptide segments as a result of the chemical ligation is an unnatural (non-peptide) bond (Schnolzer et al. Science, 256:221 (1992)). This technique has been used to synthesize analogs of protein domains as well as large amounts of relatively pure proteins with full biological activity (deLisle Milton et al. Techniques in Protein Chemistry IV, Academic Press, New York, pp. 257-267 (1992)).

The present invention also provides isolated nucleic acid molecules that encode a GPR motif, for example, SEQ ID NO: 1, or fragments thereof, as well as nucleic acid fragments sufficient for use as hybridization probes to identify GPR motifs or nucleic acids comprising a GPR motif. Additionally, nucleic acids that encode polypeptides comprising SEQ ID NO: 1 are provided.

As used herein, the term “nucleic acid” refers to single-or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the novel genes discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides).

The nucleic acids provided for by the present invention may be obtained in any number of ways. For example, a nucleic acid database can be searched for molecules that comprise a nucleotide sequence that encodes a GPR motif of the invention. If any nucleic acids are identified by this method, they have been provided by routine methods based on the disclosure of SEQ ID NO: 1.

A DNA molecule encoding a GPR motif can also be isolated from the organism in which it is normally found. For example, a genomic DNA or cDNA library can be constructed and screened for the presence of the nucleic acid of interest. Methods of constructing and screening such libraries are well known in the art and kits for performing the construction and screening steps are commercially available (for example, Stratagene Cloning Systems, La Jolla, Calif.). Once isolated, the nucleic acid can be directly cloned into an appropriate vector, or if necessary, be modified to facilitate the subsequent cloning steps. Such modification steps are routine, an example of which is the addition of oligonucleotide linkers which contain restriction sites to the termini of the nucleic acid. General methods are set forth in Sambrook et al., “Molecular Cloning, a Laboratory Manual,” Cold Spring Harbor Laboratory Press (1989).

Once the nucleic acid sequence of the desired GPR motif is obtained, the sequence encoding specific amino acids can be modified or changed at any particular amino acid position by techniques well known in the art. For example, PCR primers can be designed which span the amino acid position or positions and which can substitute any amino acid for another amino acid. Then a nucleic acid can be amplified and inserted into the wild-type GPR motif coding sequence in order to obtain any of a number of possible combinations of amino acids at any position of the GPR motif. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991). Techniques such as these can be used to alter the coding sequence without altering the amino acid sequence that is encoded.

Another example of a method of obtaining a DNA molecule encoding a GPR motif is to synthesize a recombinant DNA molecule which encodes the GPR motif. For example, oligonucleotide synthesis procedures are routine in the art and oligonucleotides coding for a particular protein region are readily obtainable through automated DNA synthesis. A nucleic acid for one strand of a double-stranded molecule can be synthesized and hybridized to its complementary strand. One can design these oligonucleotides such that the resulting double-stranded molecule has either internal restriction sites or appropriate 5′ or 3′ overhangs at the termini for cloning into an appropriate vector. Double-stranded molecules coding for relatively large proteins can readily be synthesized by first constructing several different double-stranded molecules that code for particular regions of the protein, followed by ligating these DNA molecules together. For example, Cunningham, et al., “Receptor and Antibody Epitopes in Human Growth Hormone Identified by Homolog-Scanning Mutagenesis,” Science, 243:1330-1336 (1989), have constructed a synthetic gene encoding the human growth hormone gene by first constructing overlapping and complementary synthetic oligonucleotides and ligating these fragments together. See also, Ferretti, et al., Proc. Nat. Acad. Sci. 82:599-603 (1986), wherein synthesis of a 1057 base pair synthetic bovine rhodopsin gene from synthetic oligonucleotides is disclosed. By constructing a GPR motif in this manner, one skilled in the art can readily obtain any particular GPR motif with desired amino acids at any particular position or positions within the GPR moitf. See also, U.S. Pat. No. 5,503,995 which describes an enzyme template reaction method of making synthetic genes. Techniques such as this are routine in the art and are well documented. These nucleic acids or fragments of a nucleic acid encoding a GPR motif can then be expressed in vivo or in vitro as discussed below.

The invention also provides for the isolated nucleic acids encoding a GPR motif in a vector suitable for expressing the nucleic acid. Once a nucleic acid encoding a particular GPR motif of interest, or a region of that nucleic acid, is constructed, modified, or isolated, that nucleic acid can then be cloned into an appropriate vector, which can direct the in vivo or in vitro synthesis of that wild-type and/or modified GPR motif. The vector is contemplated to have the necessary functional elements that direct and regulate transcription of the inserted gene, or nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.).

There are numerous E. coli (Escherichia coli) expression vectors known to one of ordinary skill in the art which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures.

Additionally, yeast expression can be used. There are several advantages to yeast expression systems. First, evidence exists that proteins produced in a yeast secretion systems exhibit correct disulfide pairing. Second, post-translational glycosylation is efficiently carried out by yeast secretory systems. The Saccharomyces cerevisiae pre-pro-alpha-factor leader region (encoded by the MF″-1 gene) is routinely used to direct protein secretion from yeast. (Brake, et al., A %-Factor-Directed Synthesis and Secretion of Mature Foreign Proteins in Saccharomyces cerevisiae. Proc. Nat. Acad. Sci., 81:4642-4646 (1984)). The leader region of pre-pro-alpha-factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha-factor leader region. This construct is then put under the control of a strong transcription promoter, such as the alcohol dehydrogenase I promoter or a glycolytic promoter. The nucleic acid coding sequence is followed by a translation termination codon which is followed by transcription termination signals. Alternatively, the nucleic acid coding sequences can be fused to a second protein coding sequence, such as Sj26 or β-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, gentamicin resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexinl, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

The invention also provides for the vectors containing the contemplated nucleic acids in a host suitable for expressing the nucleic acids. The vectors containing the nucleic acid segments of interest can be transferred into host cells by well-known methods, which vary depending on the type of cellular host. For example, calcium chloride transformation, transduction, and electroporation are commonly utilized for prokaryotic cells, whereas calcium phosphate, DEAE dextran, or lipofection mediated transfection or electroporation may be used for other cellular hosts.

Alternatively, the nucleic acids of the present invention can be operatively linked to one or more of the functional elements that direct and regulate transcription of the inserted nucleic acid and the nucleic acid can be expressed. For example, a nucleic acid can be operatively linked to a bacterial or phage promoter and used to direct the transcription of the nucleic acid in vitro. A further example includes using a nucleic acid provided herein in a coupled transcription-translation system where the nucleic acid directs transcription and the RNA thereby produced is used as a template for translation to produce a polypeptide. One skilled in the art will appreciate that the products of these reactions can be used in many applications such as using labeled RNAs as probes and using polypeptides to generate antibodies or in a procedure where the polypeptides are being administered to a cell or a subject.

Expression of the nucleic acid, in combination with a vector, can be by either in vivo or in vitro. In vivo synthesis comprises transforming prokaryotic or eukaryotic cells that can serve as host cells for the vector. Alternatively, expression of the nucleic acid can occur in an in vitro expression system. For example, in vitro transcription systems are commercially available which are routinely used to synthesize relatively large amounts of mRNA. In such in vitro transcription systems, the nucleic acid encoding a GPR motif would be cloned into an expression vector adjacent to a transcription promoter. For example, the Bluescript II cloning and expression vectors contain multiple cloning sites which are flanked by strong prokaryotic transcription promoters. (Stratagene Cloning Systems, La Jolla, Calif.). Kits are available which contain all the necessary reagents for in vitro synthesis of an RNA from a DNA template such as the Bluescript vectors. (Stratagene Cloning Systems, La Jolla, Calif.). RNA produced in vitro by a system such as this can then be translated in vitro to produce the desired DRL protein. (Stratagene Cloning Systems, La Jolla, Calif.).

Additionally contemplated by the present invention are nucleic acids, from any desired species, preferably mammalian and more preferably human, having 99.9%, 99.7%, 99.5%, 99.3%, 99%, 98%, 95%, 90%, 85%, 80%, 70%, 60%, or 50% homology, or less, in the region being compared to the same region of a gene comprising a nucleic acid that encodes a polypeptide as set forth in SEQ ID NO: 1 or to an isolated nucleic acid consisting of the nucleic acid encoding a polypeptide as set forth in SEQ ID NO: 1, or to allelic variants or homologs thereof. These genes and nucleic acids can be synthesized or obtained by the same methods used to isolate homologs, with stringency of hybridization and washing, if desired, reduced accordingly as homology desired is decreased, and further, depending upon the G-C or A-T richness of any area wherein variability is searched for. Allelic variants of any of the nucleic acids or of their homologs can readily be isolated and sequenced by screening additional libraries following the examples given herein and procedures well known in the art.

The present invention additionally provides a nucleic acid that selectively hybridizes under stringent conditions with a gene comprising a nucleic acid that encodes the polypeptide set forth in SEQ ID NO: 1. This hybridization can be specific. The degree of complementarity between the hybridizing nucleic acid and the sequence to which it hybridizes should be at least enough to exclude hybridization with a nucleic acid encoding an unrelated protein. Thus, a nucleic acid that selectively hybridizes with a nucleic acid of a present protein coding sequence will not selectively hybridize under stringent conditions with a nucleic acid for a different, unrelated protein, and vice versa. The temperature and salt conditions are readily determined empirically in preliminary experiments in which samples of reference DNA immobilized on filters are hybridized to a labeled nucleic acid of interest and then washed under conditions of different stringencies.

Hybridization temperatures are typically higher for DNA-RNA and RNA-RNA hybridizations. The washing temperatures can be used as described above to achieve selective stringency, as is known in the art. (See, for example, Sambrook et al., “Molecular Cloning: A Laboratory Manual” 2nd Ed., Cold Spring Harbor Laboratory, Cold Spring Harbor, N.Y. (1989) and Kunkel et al. Methods Enzymol. 1987:154:367 (1987)). Nucleic acid fragments that selectively hybridize to any given nucleic acid can be used, e.g., as primers and or probes for further hybridization or for amplification methods (e.g., polymerase chain reaction (PCR), ligase chain reaction (LCR)).

The gene or nucleic acid encoding any selected protein of the present invention can be any nucleic acid that functionally encodes that protein. A nucleic acid encoding a selected protein can readily be determined based upon the amino acid sequence of the selected protein, and, clearly, many nucleic acids will encode any selected protein.

The present invention also contemplates polynucleotide probes for detecting the GPR motif, wherein the polynucleotide probe hybridizes to the nucleotide sequence that encodes the polypeptide set forth in the Sequence Listing as SEQ ID NO: 1.

As used herein, the term “polynucleotide probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein.

Thus, allelic variants can be identified and isolated by nucleic acid hybridization techniques. Probes selective to the nucleic acid that encodes the polypeptide set forth in the Sequence Listing as SEQ ID NO: 1 can be synthesized and used to probe the nucleic acid from various cells, tissues, libraries etc. High sequence complementarity and stringent hybridization conditions can be selected such that the probe selectively hybridizes to allelic variants of the nucleic acid sequence that encodes the polypeptide set forth in the Sequence Listing as SEQ ID NO:1. For example, the selectively hybridizing nucleic acids of the invention can have at least 70%, 80%, 85%, 90%, 95%, 97%, 98% and 99% complementarity with the segment of the sequence to which it hybridizes. The nucleic acids can be at least 12, 50, 100, 150, 200, 300, 500, 750, or 1000 nucleotides in length. Thus, the nucleic acid can be a coding sequence for the GPR motif or fragments thereof that can be used as a probe or primer for detecting the presence of these genes. If used as primers, the invention provides compositions including at least two nucleic acids which hybridize with different regions so as to amplify a desired region.

“Stringent conditions” refers to the washing conditions used in a hybridization protocol. In general, the washing conditions should be a combination of temperature and salt concentration chosen so that the denaturation temperature is approximately 5-20° C. below the calculated Tm of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tₘ of such an oligonucleotide can be estimated by allowing 2° C. for each A or T nucleotide, and 4° C. for each G or C. For example, an 18 nucleotide probe of 50% G+C would, therefore, have an approximate Tₘ of 54° C.

The present invention also contemplates any unique fragment of these nucleic acids that encode the polypeptide set forth in SEQ ID NO:1. To be unique, the fragment must be of sufficient size to distinguish it from other known sequences, most readily determined by comparing any nucleic acid fragment to the nucleotide sequences of nucleic acids in computer databases, such as GenBank. Such comparative searches are standard in the art. Typically, a unique fragment useful as a primer or probe will be at least about 20 to about 25 nucleotides in length, depending upon the specific nucleotide content of the sequence. Additionally, fragments can be, for example, at least about 30, 40, 50, 75, 100, 200, 300, 400, 500, 600, 700, or 800 nucleotides in length. All of nucleic acids, and fragments of the nucleic acids disclosed and contemplated herein can be single or double stranded, depending upon the purpose for which it is intended.

The invention also contemplates compounds comprising the nucleic acids, and fragments of the nucleic acids as disclosed and contemplated herein. For example, a compound comprising a nucleic acid can be a derivative of a typical nucleic acid such as nucleic acids which are modified to contain a terminal or internal reporter molecule and/or those nucleic acids containing non-typical bases or sugars. These reporter molecules include, but are not limited to, isotopic and non-isotopic reporters. Examples include, a FLAG tag or a human IgG Fc. Therefore any molecule which may aid in detection, amplification, replication, expression, purification, uptake, etc. may be added to the nucleic acid construct.

The present invention also provides a ligand that specifically binds to the polypeptide set forth in the Sequence Listing as SEQ ID NO: 1. As used herein, a ligand may be an antibody, a compound such as a chemical or small molecule that binds to a GPR motif, a G-protein subunit or a fragment of a G-protein subunit that binds to the GPR motif, a protein or a fragment of a protein that interacts with the GPR motif during signal transduction or in any other activity modulated by the GPR motif or a protein comprising the GPR motif.

Also provided herein are purified antibodies that selectively or specifically bind to the GPR polypeptides provided and contemplated herein, for example, purified antibodies which selectively or specifically bind to the polypeptide set forth in SEQ ID NO: 1. The antibody (either polyclonal or monoclonal) can be raised to any of the polypeptides provided and contemplated herein, both naturally occurring and recombinant polypeptides, and immunogenic fragments, thereof. The antibody can be used in techniques or procedures such as diagnostics, treatment, or vaccination. Anti-idiotypic antibodies and affinity matured antibodies are also considered.

Antibodies can be made by many well-known methods (See, e.g. Harlow and Lane, “Antibodies; A Laboratory Manual” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al. Bio/Technology, 10: 163-167 (1992); Bebbington et al. Bio/Technology, 10: 169-175 (1992)). Humanized and chimeric antibodies are also contemplated in this invention. Heterologous antibodies can be made by well known methods (See, for example, U.S. Pat. Nos. 5,545,806, 5,569,825, 5,625,126, 5,633,425, 5,661,016, 5,770,429, 5,789,650, and 5,814,318)

The phrase “specifically binds” with the polypeptide refers to a binding reaction which is determinative of the presence of the protein in a heterogeneous population of proteins and other biologics. Thus, under designated immunoassay conditions, the specified antibodies bound to a particular protein do not bind in a significant amount to other proteins present in the sample. Selective binding to an antibody under such conditions may require an antibody that is selected for its specificity for a particular protein. A variety of immunoassay formats may be used to select antibodies that selectively bind with a particular protein. For example, solid-phase ELISA immunoassays are routinely used to select antibodies selectively immunoreactive with a protein. See Harlow and Lane “Antibodies, A Laboratory Manual” Cold Spring Harbor Publications, New York, (1988), for a description of immunoassay formats and conditions that could be used to determine selective binding.

The invention also provides a method of identifying a test polypeptide as a putative receptor-independent activator of G-protein signaling pathways comprising detecting SEQ ID NO: 1 in a test polypeptide, wherein the presence of SEQ ID NO: 1 in the test polypeptide indicates the test polypeptide is a putative receptor-independent activator of heterotrimeric G-protein signaling pathways.

For example, the GPR motif or fragments thereof can be utilized to search databases such as GenBank for other G-protein modulator proteins (GPM) that contain the GPR motif. A putative GPM protein can be identified by searching databases for nucleic acids that encode a GPR motif such as SEQ ID NO:1 or by searching databases for polypeptides that contain a GPR motif. The putative GPM proteins identified via this method can then be easily assessed for receptor independent activation of G-protein signal transduction using, for example, the yeast system described in the Examples. If the putative GPM protein effects receptor independent activation of G-protein signal transduction or effects any other activity of a GPR protein, the putative GPR protein can be classified as a GPM protein.

A putative GPM protein can also be identified by isolating a nucleic acid, sequencing the nucleic acid, translating the nucleic acid sequence into a polypeptide sequence and identifying SEQ ID NO: 1 in the translated polypeptide sequence encoded by the isolated nucleic acid. The GPR motif of this invention can also be isolated.

An analog of a polypeptide consisting of SEQ ID NO: 1 is provided as is an analog of a polypeptide comprising SEQ ID NO: 1. An “analog” is any compound structurally similar to SEQ ID NO: 1 which has an activity of SEQ ID NO: 1. The GPR motif, an example of which is SEQ ID NO: 1, may interact with G-proteins and regulate subunit interactions, nucleotide exchange, GTP hydrolysis and/or interaction with a G-protein with other entities involved in signal transduction. Mimetics of a GPR motif such as SEQ ID NO: 1 or of a peptide comprising a GPR motif such as SEQ ID NO: 1 are also provided. Mimetics include, for example, peptides, compounds and small molecules which mimic the function of these GPR motifs although they need not be structurally related. Mimetics of a GPR motif such as SEQ ID NO: 1 or of a polypeptide comprising a GPR motif can have activating or stimulating effects as well as inhibitory effects such as those described in Example II. Therefore, the present invention provides methods of screening for mimetics of a GPR motif as well as methods of screening for a mimetic of a polypeptide comprising a GPR motif.

Also provided is method of screening for a mimetic or analog of SEQ ID NO: 1 or a fragment thereof, comprising the steps of a) contacting a model system known to express an activity of SEQ ID NO: 1 with a putative agonist analog; b) detecting the presence or absence of the known activity of SEQ ID NO: 1 in the presence of the putative mimetic; and c) correlating the presence or absence of the activity with the presence of a mimetic of SEQ ID NO: 1.

As described above, the activity of a GPR motif, such as SEQ ID NO: 1 can be a stimulating activity or an inhibitory activity. Therefore, in the methods of the present invention, the known activity of a GPR motif, such as SEQ ID NO: 1, can be a, stimulating activity or an inhibitory activity. In the methods of the present invention, a stimulating activity can be, but is not limited to, the activation of G-protein mediated signal transduction. The inhibitory activity can be, but is not limited to, the inhibition of G protein mediated signal transduction. Thus, in screening for a mimetic or analog of a GPR motif, the mimetic can mimic a stimulating activity or an inhibitory activity. For example, if a particular GPR motif has stimulatory activity in a model system, such as those described in the Examples, a potential mimetic can be tested in the same system. If the stimulatory activity of the GPR motif is observed in the presence of the putative mimetic, the putative mimetic is a mimetic of the GPR motif possessing stimulatory activity. Similarly, if a particular GPR motif has inhibitory activity, a potential mimetic can be tested in the same system. If the inhibitory activity of the GPR motif is observed in the presence of the putative mimetic, the putative mimetic is a mimetic of the GPR motif possessing inhibitory activity.

A purified antagonist of the activity of a GPR motif such as SEQ ID NO: 1 is also provided. An “antagonist” is defined as a compound that binds SEQ ID NO: 1 or a compound, including antibodies, that binds the target for SEQ ID NO: 1 and prevents an activity of SEQ ID NO: 1. Non-antibody antagonists are also provided. Antibody and non-antibody antagonists can be used in the methods of the invention. Such compounds most likely bind SEQ ID NO: 1 or other such molecular species such as a G-protein subunit with which SEQ ID NO: 1 or its analogs combine, and prevent or reduce the interaction between SEQ ID NO: 1 and a G-protein subunit.

Thus, a method of screening for an antagonist of SEQ ID NO: 1 is provided, comprising: a) contacting a model system known to express an activity of a polypeptide comprising a GPR motif with a putative antagonist in the presence of SEQ ID NO: 1 or a fragment thereof; b)detecting the absence of, or reduction in, the known activity of the GPR motif, in the presence of the putative antagonist; c) correlating the absence of, or reduction in, the activity with the presence of an antagonist of SEQ ID NO: 1 or a fragment thereof and d) confirming that the antagonist interacts with SEQ ID NO: 1 or interferes with the binding of SEQ ID NO: 1 to a G-protein.

As described above, the activity of a polypeptide comprising a GPR motif, such as SEQ ID NO: 1, can be a stimulating activity or an inhibitory activity. Therefore, in the methods of the present invention, the known activity of a polypeptide comprising a GPR motif or the known activity of a GPR motif, such as SEQ ID NO: 1, can be a stimulating activity or an inhibitory activity. Thus, in screening for an antagonist of a GPR motif, the antagonist can block or antagonize a stimulating activity or the antagonist can block or antagonize an inhibitory activity. Therefore, an antagonist of a GPR motif can prevent stimulation by a polypeptide comprising a GPR motif that is a stimulating polypeptide or prevent inhibition by a polypeptide comprising a GPR motif that is an inhibitory polypeptide.

In order to determine whether or not a compound, for example, an antagonist, interacts with SEQ ID NO: 1 one of skill in the art can perform competitive binding assays known in the art that can determine whether or not the antagonist interferes with the binding of an antibody to SEQ ID NO: 1. Also contemplated are assays where allosteric interactions between a compound and a polypeptide comprising SEQ ID NO: 1 can be identified. For example, a compound might be identified that binds to amino acids of the polypeptide comprising SEQ ID NO: 1 which alters the conformation of the SEQ ID NO: 1 and subsequently, does not allow the SEQ ID NO: 1 to interact with its target, such as a G-protein subunit. Also contemplated by this invention are compounds which bind to a polypeptide comprising SEQ ID NO: 1 to alter the conformation of SEQ ID NO: 1 in such a way as to enhance the interaction between SEQ ID NO: 1 and its target, such as a G-protein subunit.

The mimetic or antagonist of the GPR motif of the present invention can be further identified as an activator or inhibitor respectively, of G-protein activation. The identification of a compound as an activator or inhibitor of G-protein activation is determined by a variety of techniques including a) increased or decreased binding of GTP and or GTP analogs to purified G-protein (Sato M, Ribas C, Hildebrandt J D and Lanier S M. Characterization of a G-protein activator in the neuroblastoma glioma cell hybrid NG108-15. J Biol Chem (1996) 271:30052-30060). b) increased or decreased GTPase activity by purified G-proteins c) increased or decreased dissociation of GDP bound to purified G-protein and/or d) alterations in the interaction of purified G-protein alpha subunit with beta/gamma subunits which can be determined by direct analysis of subunit interactions by gel filtration or by analysis of functional phosphorylation of a substrate such a GPCR subdomain (Wu G, Hildebrandt J, Benovic J L and Lanier S M. Receptor docking sites for Gβγ: implications for signal propagation (1998) (Communication) J Biol Chem. 273:7197-7200.) In the latter case, free Gbeta gamma is required for GRK2 mediated phosphorylation of receptor subdomains and this is inhibited by Galpha subunit or possibly by the compound.

Another path of analysis is to determine the binding of the polypeptide (s) to G-protein subunits as described in detail (Takesono A, Cismowski M, Michael Bernard, Catalina Ribas, Peter Chung, Duzic E and Lanier S M. Receptor-independent activators of heterotrimeric G-protein signalling. (1999) (Communication) J Biol Chem.274: 33202-33205; Wu G, Hildebrandt J, Benovic J L and Lanier S M. Receptor docking sites for Gbg: implications for signal propagation (1998) (Communication) J Biol Chem. 273:7197-7200.

The influences of a compound on guanine nucleotide binding to brain Go in the solution phase is determined as previously described (Sato et al., 1996). Briefly, a preincubation cocktail was prepared for each assay point. Heterotrimeric G-proteins were preincubated with or without the partially purified material from NG10815 cells for 1 hr at 4° C. Preincubation cocktail=5 μM GDP, 6.25 nM brain G-protein or 50 nM transducin. 15 μl of a test compound in a total volume of 25 ul with final buffer concentrations of 0.066% Thesit, 50 mM Tris-HCl Ph.7.4, 5 mM MgCl₂, 0.6 mM EDTA. After this preincubation period, 10 μl of this cocktail for each sample was added to 40 μl assay mixture containing GTPγ³⁵S. The final buffer composition in the assay mixture tube was 50 mM Tris-HCl pH 7.4, 0.6 mM EDTA, 5 mM MgCl₂, 150 mM NaCl, 1 mM DTT, ˜230,000 cpm (˜2 nM) GTPγ³⁵S, 1 uM GDP and 0.013% Thesit. Incubation was then continued for 30 minutes unless otherwise indicated at 24° C. and terminated by vacuum filtration on BA85 (S & S) nitrocellulose membranes with 4×4-ml washes (50 mM Tris-HCl, 5 mM MgCl₂, pH 7.4, 4° C.). Radioactivity bound to the filters was determined by liquid scintillation counting. Nonspecific binding was defined with 10 uM GTPgS. Each assay actually contained three sets of preincubation tubes: 1) G-protein without the test compound, 2) test compound without G-protein, 3) G-protein plus test compound. Thus, in a cell extract containing a putative “G-protein activator” the nucleotide bound in set one would be greater than the sum of sets one and two. In a cell extract containing a putative “G-protein inhibitor” the nucleotide bound in set one would be less than the sum of sets one and two.

The present invention also provides a method of identifying a compound that inhibits the interaction of a polypeptide comprising SEQ ID NO: 1 and a G-protein subunit comprising: contacting the polypeptide comprising SEQ ID NO: 1 with a G-protein subunit, administering the compound, determining whether the compound inhibits the interaction of the polypeptide comprising SEQ ID NO: 1 and the G-protein subunit; and determining whether the compound interacts with SEQ ID NO:1 or with a polypeptide comprising the polypeptide set forth in SEQ ID NO: 1, thereby identifying a compound that inhibits the interaction of the polypeptide of and a G-protein subunit. In the method described above, the polypeptide comprising SEQ ID NO: 1 can be contacted with the a G-protein subunit and a compound simultaneously, or the polypeptide comprising SEQ ID NO: 1 can be contacted with the compound prior to contacting the polypeptide comprising SEQ ID NO: 1 with a G-protein subunit.

The cell-free G-protein assay set forth in the Examples and elsewhere in the application can be utilized to determine whether the compound interacts with SEQ ID NO: 1 or a region of a G-protein subunit that interacts with SEQ ID NO: 1.

The present invention also provides a method of identifying a compound that inhibits the interaction of a polypeptide comprising SEQ ID NO: 1 and a G-protein comprising, expressing the polypeptide in a cell; detecting receptor independent activation of G-protein signaling; administering a compound to the cell; detecting inhibition of receptor independent activation of G-protein signaling; and determining whether the compound interacts with SEQ ID NO: 1 of the polypeptide, thereby identifying a compound that inhibits the interaction of the polypeptide comprising SEQ ID NO: 1 and a G-protein.

In order to determine whether or not a compound interacts with SEQ ID NO: 1, SEQ ID NO: 1 or fragments of SEQ ID NO: 1 can be immobilized by biotinylating SEQ ID NO: 1 and immobilizing the peptide in wells on streptavidin coated plates. The compound is added to the wells and allowed to form a complex with the immobilized peptide. Immunodetection techniques known in the art can be utilized to detect the complexes. For example, an antibody to SEQ ID NO: 1 or an antibody to the compound can be utilized to detect complexes formed between SEQ ID NO: 1 and the compound.

The invention further provides a method of inhibiting the interaction of the polypeptide comprising SEQ ID NO: 1 and a G-protein, comprising administering to a subject a ligand that specifically binds to amino acids in the sequence set forth as SEQ ID NO: 1.

The invention further provides a method of stimulating G-protein mediated signal transduction comprising administering to a subject a mimetic of a GPR motif. An example of such a GPR motif is a polypeptide consisting of or comprising SEQ ID NO: 1.

The term “stimulating” is familiar to one skilled in the art and is used herein to describe any compound or composition which stimulates or increases G-protein mediated signal transduction. The degree of stimulation comprises any stimulation of G-protein mediated signal transduction relative to G-protein mediated signal transduction in a similar environment in the absence of the inhibiting compound.

The invention further provides a method of inhibiting G protein mediated signal transduction comprising administering to a subject a GPR motif or mimetic of a GPR motif.

The term “inhibiting” is familiar to one skilled in the art and is used herein to describe any compound or composition which inhibits or decreases G-protein mediated signal transduction. The degree of inhibition does not have to be complete, such as completely inhibiting signal transduction and therefore comprises any inhibition of G-protein mediated signal transduction relative to G-protein mediated signal transduction in a similar environment in the absence of the inhibiting compound. Inhibition can occur in many ways such as by acting as a guanine nucleotide dissociation inhibitor or by blocking receptor coupling. As described in Example II, inhibition can occur by inhibiting guanine nucleotide dissociation of Giα or by blocking receptor coupling to Giαβγ.

The subject which can be treated by the methods of this invention can be any animal. In a preferred embodiment, the animal of the present invention is a human. In addition, non-human animals which can be treated by the methods of this invention can include, but are not limited to, cats, dogs, birds, horses, cows, goats, sheep, guinea pigs, hamsters, gerbils and rabbits.

In the present invention, the polypeptide comprising SEQ ID NO: 1, the polypeptide consisting of SEQ ID NO: 1, an antibody to a polypeptide comprising SEQ ID NO: 1, or a fragment of SEQ ID NO: 1 can be orally or parenterally administered in a carrier pharmaceutically acceptable to human subjects. Suitable carriers for oral or inhaled administration can include one or more of the carriers pharmaceutically acceptable to human subjects. Suitable carriers for oral administration include one or more substances which may also act as a flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pyrogen free saline, pharmaceutically accepted oils, or a mixture of any of these. The liquid can also contain other suitable pharmaceutical addition such as buffers, preservatives, flavoring agents, viscosity or osmo-regulators, stabilizers or suspending agents. Examples of suitable liquid carriers include water with or without various additives, including carboxypolymethylene as a ph-regulated gel. The GRP motif may be contained in enteric coated capsules that release the polypeptide into the intestine to avoid gastric breakdown. For parenteral administration of the antagonist, a sterile solution or suspension is prepared in saline that may contain additives, such as ethyl oleate or isopropyl myristate, and can be injected for example, into subcutaneous or intramuscular tissues, as well as intravenously.

Similarly, using gene therapy methods, a nucleic acid encoding a polypeptide comprising SEQ ID NO: 1 or functional fragment can be administered. The nucleic acid encoding the peptide of this invention can be placed into a vector and delivered to the cells of a subject either in vivo or ex vivo by standard methods.

For in vivo administration, the cells can be in a subject and the nucleic acid can be administered in a pharmaceutically acceptable carrier. The subject can be any animal in which it is desirable to selectively express a nucleic acid in a cell. In a preferred embodiment, the animal of the present invention is a human. In addition, non-human animals which can be treated by the method of this invention can include, but are not limited to, cats, dogs, birds, horses, cows, goats, sheep, guinea pigs, hamsters, gerbils and rabbits, as well as any other animal in which selective expression of a nucleic acid in a cell can be carried out according to the methods described herein.

In the method described above which includes the introduction of exogenous DNA into the cells of a subject (i.e., gene transduction or transfection), the nucleic acids of the present invention can be in the form of naked DNA or the nucleic acids can be in a vector for delivering the nucleic acids to the cells for expression of the nucleic acid inside the cell. The vector can be a conumercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as Lipofectin®, Lipofectamine® (GIBCO-BRL, Inc., Gaithersburg, Md.), Superfect® (Qiagen, Inc. Hilden, Germany) and Transfectam® (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a Sonoporation machine (ImaRx Pharmaceutical Corp., Tucson, Ariz.).

As one example, vector delivery can be via a viral system, such as a retroviral vector system which can package a recombinant retroviral genome. The recombinant retrovirus can then be used to infect and thereby deliver nucleic acid to the infected cells. The exact method of introducing the nucleic acid into mammalian cells is, of course, not limited to the use of retroviral vectors. Other techniques are widely available for this procedure including the use of adenoviral vectors, adeno-associated viral (AAV) vectors, lentiviral vectors, pseudotyped retroviral vectors, and pox virus vectors, such as vaccinia virus vectors. Physical transduction techniques can also be used, such as liposome delivery and receptor-mediated and other endocytosis mechanism. This invention can be used in conjunction with any of these or other commonly used gene transfer methods.

The nucleic acid and the nucleic acid delivery vehicles of this invention, (e.g., viruses; liposomes, plasmids, vectors) can be in a pharmaceutically acceptable carrier for in vivo administration to a subject. By “pharmaceutically acceptable” is meant a material that is not biologically or otherwise undesirable, i.e., the material may be administered to a subject, along with the nucleic acid or vehicle, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The nucleic acid or vehicle may be administered orally, parenterally (e.g., intravenously), by intramuscular injection, by intraperitoneal injection, transdermally, extracorporeally, topically or the like. The exact amount of the nucleic acid or vector required will vary from subject to subject, depending on the species, age, weight and general condition of the subject, the severity or mechanism of any disorder being treated, the particular nucleic acid or vehicle used, its mode of administration and the like.

Alternatively, an antisense molecule to the mRNA of a polypeptide comprising a GPR motif can be administered. Antisense technology is well known in the art and describes a mechanism whereby a nucleic acid comprising a nucleotide sequence which is in a complementary, “antisense” orientation with respect to a coding or “sense” sequence of an endogenous gene is introduced into a cell, whereby a duplex forms between the antisense sequence and its complementary sense sequence. The formation of this duplex results in inactivation of the endogenous gene. Antisense nucleic acid can be produced for any endogenous gene for which the coding sequence has been or can be determined according to well known methods.

Antisense nucleic acid can inhibit gene expression by forming an RNA/RNA duplex between the antisense RNA and the RNA transcribed from a target gene. The precise mechanism by which this duplex formation decreases the production of the protein encoded by the endogenous gene most likely involves binding of complementary regions of the normal sense mRNA and the antisense RNA strand with duplex formation in a manner that blocks RNA processing and translation. Alternative mechanisms include the formation of a triplex between the antisense RNA and duplex DNA or the formation of a DNA-RNA duplex with subsequent degradation of DNA-RNA hybrids by RNAse H. Furthermore, an antisense effect can result from certain DNA-based oligonucleotides via triple-helix formation between the oligomer and double-stranded DNA which results in the repression of gene transcription.

The polypeptides, nucleic acids, antibodies, mimetics, and other molecules contemplated by this invention can be utilized to treat disorders associated with G-protein coupled receptors such as Albright hereditary osteodystrophy, nephrogenic diabetes insipidus, McCune-Albright syndrome, and familial male precocious puberty to name a few (Speigel, A M Inborn errors of signal transduction: mutations in G proteins and G protein-coupled receptors as a cause of disease. J Inherit Metab Dis Jun. 20, 1997 (2):113-21).

The present invention is more particularly described in the following examples which are intended as illustrative only since numerous modifications and variations therein will be apparent to those skilled in the art.

### EXAMPLES

Generation and screening of cDNA libraries. The NG108-15 cell line was propagated as previously described (4). mRNA was prepared from twenty 100 mm confluent plates of cells and used to generate a cDNA library in the yeast expression vector pYES2. The library was constructed by standard procedures using reagents from Stratagene. Analysis of individual transformants indicated cDNA inserts ranging from 400 to 3000 nt with ˜70-80% of the transformants containing cDNA inserts. The primary transformants were then amplified by solid phase agarose growth for 48 hr and plasmid DNA isolated by column fractionation (Qiagen). The NG108-15 cDNA library was then screened for activators of the pheromone response pathway as described (5). We used a his3 far1 yeast strain containing a genomic integration of the FUS1p-HIS3 reporter construct and lacking both the native pheromone response receptor Ste3 and Gpa1 Gα subunit. A plasmid carrying a modified mammalian Giα2 subunit in which the amino terminus was replaced with the first 41 amino acids of Gpa1 was introduced into this strain. Essentially, the screen took advantage of the inducible expression of the library cDNAs by differential plating of transformants on selective media. Galactose was added to the selective medium to induce expression of cDNAs cloned into the pYES2 vector. Thus, a cDNA whose expressed protein product was capable of activating the pheromone response pathway would confer growth on selective medium. Transformants exhibiting growth in the primary screen were rescreened for galactose-dependent growth by spot growth assays. Transformants identified as positive in the second screen were then further propagated to isolate plasmid DNA. The isolated plasmid was then used to retransform yeast and verify that the cDNA conferred galactose-dependent growth in the selective media. The cDNAs conferring such a response were considered positive and were further evaluated by epistasis experiments in different yeast strains, nucleotide sequencing and RNA blot analysis. Yeast growth assays, yeast strain constructions and β-galactosidase reporter assays were conducted as described (5, 7).

cDNA analysis—A rat brain cDNA library was screened with a ³²P labeled 43 mer oligonucleotide (5′ ATAAGGCTGAAGAAATCCTCATCAGGCATGGTAGGGCCAC 3′ (SEQ ID NO:2)) derived from the AGS3 sequence cDNA isolated in the yeast screen. 800,000 individual plaques were screened by filter transfer/hybridization exactly as previously described (8). The longest brain AGS3 cDNA still contained a truncated reading frame and the 5′ end was extended by 5′RACE using Marathon-Ready cDNA (CLONTECH) from rat brain. Dideoxy nucleotide sequencing was performed in the MUSC DNA sequencing facility. Sequence retrieval and analyses were conducted via Genetics Computer Group version 9.1 software licensed to the Medical University of South Carolina and funded through the Biomolecular Computing Resource of the University's Research Resource Facilities. Multiple alignments were performed by the GCG PILEUP program using default settings for gap insertion and elongation. Domain searches were performed by the GCG COMPARE/DOTPLOT PROFILESEARCH and MOTIFS commands, via internet access to the Simple Modular Architecture Research Tool (SMART) databases (9). Multiple Expectation Maximization for motif Elucidation version 2.2 (MEME), Motif Alignment and Search Tool (MAST) and Profile searches were performed via web resources at the San Diego SuperComputer Center (10). Secondary structure analysis was performed with the GCG PEPPLOT, HELICALWHEEL and MOMENT programs as well as by the PROBE program and the PredictProtein network server at EMBL-Heidelberg (11, 12). Both AGS2 and AGS3 were evaluated by SMART analysis, the PROBE program or the UCLA/DOE Fold Prediction server to identify potential functional domains.

Immunoblotting and G-protein interaction assays. Protein interaction assays with purified G-protein subunits and immunoblotting were conducted as previously described (2). GST fusion proteins were generated in bacteria and purified on a glutathione affinity matrix as described (2). For protein interaction studies in crude cell lysates, confluent 100 mm dishes of DDT1-MF2 cells were lysed in 50 mM Tris HCl pH 7.4, 5 mM EDTA, 150 mM NaCl, 1 mM phenylmethyl sulfonyl fluoride, 60 uM benzamidine, 40 uM pepstatin, 1% NP-40 (500 ul/dish) and centrifuged at 14,000×g. For interaction assays, 225 ul of lysate containing GDP (30 uM) or GTPγS (30 uM) were incubated with GST or GST-AGS3 (300 nM) for 30 minutes at 24° C. or at 4° C. for 12 hrs. 25 ul of a 50% slurry of glutathione-sepharose was added and the incubation continued for 1 hr. Samples were then centrifuged and the matrix washed with 2×300 ul of lysis buffer prior to solubilization and denaturing gel electrophoresis as described (2). For immunoprecipitation, antisera generated against the C-terminus of Gi3 (1 to 50 dilution of sera) was incubated with 250 ul of precleared lysate for 16 hrs at 4° C. and immune complexes isolated on gammabind G sepharose. Bound proteins were detected by immunoblotting membrane transfers with specific antisera. The peptide C-VDLAGSPEQEASGLPDPQQQYPPGAS (SEQ ID NO:3), used for generation of AGS3 antisera, was synthesized in the MUSC peptide synthesis facility and rabbits immunized through the MUSC antibody production facility.

Protein interaction assays. Purified proteins (˜5 ug) immobilized on a glutathione resin (2-10 ul of packed resin) are incubated with G-protein in a total volume of 250 ul of buffer A (20 mM Tris-HCl, pH 7.5, 0.6 mM EDTA, 1 mM DTT, 70 mM NaCl, 0.01% Thesit) at 4° C. for 90 min with gentle rotation. The resin was washed three times with 0.5 ml of buffer A at 4° C. and the retained proteins were solubilized in Laemmli sample buffer and applied to a denaturing 10% polyacrylamide gel. PVDF membrane transfers were evaluated by immunoblotting. Additional details on protein interaction assays are found in Wu G, Krupnick J G, Benovic J L and Lanier S M. Interaction of arrestins with intracellular domains of receptors coupled to heterotrimeric G-proteins. J Biol Chem (1997) 272:17836-17842.

Phosphorylation of the M3-MR i3 peptide by GRK2—The incubation conditions for phosphorylation reactions were essentially as previously described for the intact M2-MR, M3-MR and b-AR (Wu G, Hildebrandt J, Benovic J L and Lanier S M. Receptor docking sites for Gbg: implications for signal propagation (1998) (Communication) J Biol Chem. 273:7197-7200). Briefly, the reaction was carried out in a total volume of 50 ul of buffer (20 mM Tris-HCl, pH 7.2, 2 mM EDTA, 7 mM MgCl2) containing 2-4 pmol of the GST-M3-MR i3 peptide fusion protein and 50 nM GRK2 with or without various additions as described in figure legends. Unless indicated otherwise, all phosphorylation reactions contained 300 uM PI. The reactions were initiated by addition of 0.1 mM [g32P]-ATP (500-1000 cpm/pmol) and incubated at 30° C. for 40 min. The reactions were stopped by addition of 50 ul of 2×Laemmli sample buffer and electrophoresed on 10% SDS-polyacrylamide gels. The gels were dried and subsequently exposed to Kodak XAR-5 film for 1 to 12 hrs. The amount of peptide phosphorylation was determined by cutting the phosphorylated bands from the gel and quantitation by liquid scintillation spectrometry.

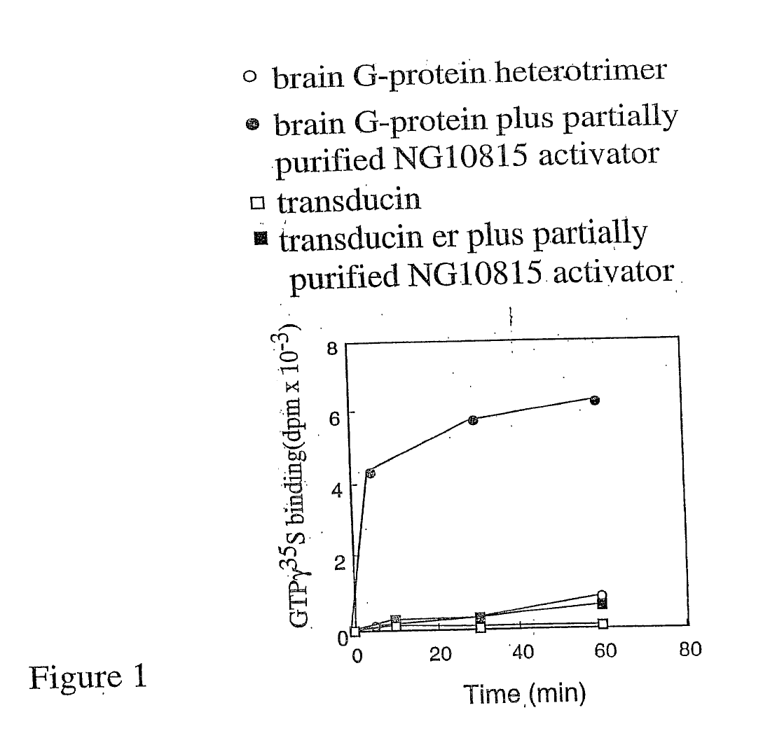

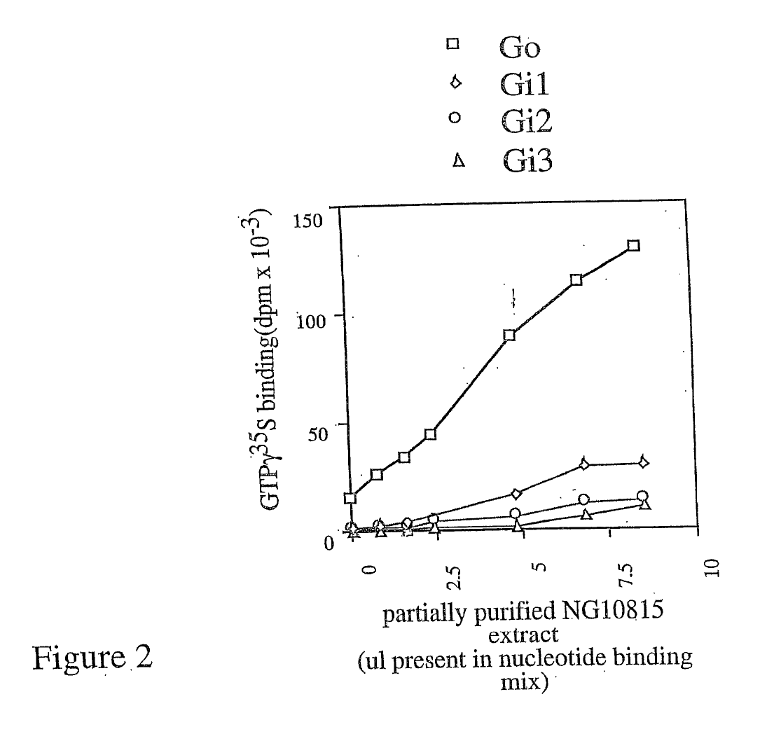

To facilitate the identification of novel receptor independent activators of heterotrimeric G-proteins, we developed an expression cloning system based upon the pheromone response pathway in Saccharomyces cerevisiae. A yeast strain that lacked the pheromone receptor and contained a modified mammalian G-protein (Giα2) in place of the yeast G-protein Gpa1 (5, 13) was utilized. The yeast strain was further modified to respond to activation of the pheromone response pathway with a readout of growth (5). As an initial source of such G-protein activators (4) a NG108-15 cDNA library was generated in the galactose-inducible yeast expression vector pYES2 for analysis in the yeast-based expression cloning system. Three rounds of transformation/screening with different NG108-15 cDNA libraries yielded three distinct cDNAs (#34, #37 and #53). Each of these cDNAs promoted growth in a galactose-dependent manner (FIGS. 1A, B). Epistasis experiments indicated that cDNA #34, which was the weakest of the three in the yeast screen, acted downstream of Ste5 (a component of the yeast MAP kinase cascade) in the pheromone response pathway and this protein will be described elsewhere. cDNAs #37 and 53 did not function in the null Ste5 genetic background and were also inactive in yeast strains lacking Ste20 (yeast homolog of PAK kinases) or Ste4 (yeast homolog of Gβ) indicating that these proteins activated the pheromone response pathway at or near the level of G-protein (FIG. 1B). Efforts were focused on cDNAs #37 and 53. Immunoblot analysis indicated that neither cDNA altered the levels of Gα or Gβ subunits. The selectivity of the two cDNAs for different G-protein heterotrimers was determined using yeast strains expressing Gpa1 (yeast Gα), Gsα, Giα2_{Gpa1(1-41)}, Giα3_{Gpa1(1-41)} and Gα16_{Gpa1(1-41)}. In β-galactosidase reporter assays, cDNA #37 was active in each of the genetic backgrounds except Gpa1, whereas cDNA #53 was only active in the Giα2 and Giα3 genetic backgrounds (FIG. 2A). The preceding observations indicated that the activation of the pheromone response pathway by cDNAs #37 and 53 depended upon the presence of heterotrimeric G-proteins and the composition of subunit isoforms. cDNAs isolated via this expression cloning system were therefore named Activators of G-protein Signalling (AGS). cDNAs #37 and 53 were termed AGS2 and AGS3, respectively. AGS1, isolated from a human liver cDNA library, encodes a novel ras-related protein and is described elsewhere (7).

Sequence analysis indicated that AGS2 (770 nt) encoded a protein identical to mouse Tctex1, which was originally isolated as a component of the mouse T complex and implicated in the generation of sterile mice. Subsequently, Tctex1 AGS2 was identified as a light chain component of the cytoplasmic motor protein dynein and the flagellar dynein inner arm (14, 15). Tctex1 also exists in the cell free of dynein where it may subserve as yet undefined roles in cellular signaling (16). Tctex1 physically associates with the G-protein coupled receptor rhodopsin, the tyrosine kinase fyn and a putative regulator of neurotransmitter release Doc2 (17-19). The functionality of AGS2 (Tctex1) in the yeast assay system also suggest a direct interaction with heterotrimeric G-proteins. This issue was addressed in protein interaction studies using a glutathione-S-transferase-AGS2 fusion protein and purified G-protein subunits. AGS2 did not interact with Giα2 but directly bound brain Gβγ (FIG. 2B). This observation is particularly interesting relative to the interaction of AGS2 with a G-protein coupled receptor (19). The bioactivity associated with AGS2 in the yeast assay system also suggests a regulatory role of heterotrimeric G-protein subunits in dynein function. Indeed, both Gα and Gβγ are implicated in various aspects of membrane trafficking, cytoskeletal dynamics and vesicular transport (20-24).

AGS3 consisted of 1,500 nt and contained a truncated open reading frame at its 5′ terminus with a methionine embedded in a Kozak's consensus sequence for translational initiation. The bioactivity/expression of this open reading frame (74 amino acids) was confirmed by mutational analysis and immunoblotting with specific peptide antisera. The AGS3 peptide was generated as a GST-fusion protein and evaluated for specific interactions with purified G-protein subunits (FIG. 2B). AGS3 specifically bound recombinant Giα2, but it did not interact with purified brain Gβγ (FIG. 2B). AGS3 preferred the GDP bound conformation of Gα_ and exhibited selectivity for Gα subunits as observed in the functional assays described above (FIGS. 2A, 2B). Thus, the two AGS proteins interacted with different mammalian G-protein subunits.

The protein interaction data and the functional data in yeast clearly indicated that AGS2 and AGS3 activated the pheromone response pathway by a process that involves heterotrimeric G-protein. As an initial approach to define their mechanism of action in the context of the cellular environment, the yeast system was utilized. Yeast strains in which signal processing in the pheromone response pathway was compromised by replacement of the Giα2 subunit with a G204A site-directed mutant Giα2 or overexpression of the GTPase activating protein RGS4. Giα2 G204A behaves as a dominant negative Gα subunit by virtue of its predicted low affinity for GTP and it is incapable of supporting signal propagation by an activated receptor in our yeast system were used.¹ Overexpression of RGS4 rapidly terminates receptor-mediated activation of the pheromone response pathway (25). Thus, if AGS2 and AGS3 were activating G-proteins by a mechanism similar to a G-protein coupled receptor, then their action would be blocked in such genetic backgrounds. Surprisingly, this was not the case as AGS2 and AGS3 were still active in the presence of the Giα2 G204A mutant or RGS4 (FIG. 2C). In contrast, another AGS protein (AGS1) isolated in the same functional screen and described elsewhere (5, 7), was not active in the same genetic backgrounds thus serving as a positive internal control. AGS2 and AGS3 also did not alter GTPγ³⁵S binding to heterotrimeric brain G-protein or Giα2, which again contrasts with the effects of AGS1 and the NG108-15 G-protein activator (4, 5, 7).¹ These data suggest that although AGS2 and AGS3 clearly activate the pheromone response way at the level of G-protein, this event does not require the generation of GαGTP. By binding to G-protein subunits, AGS2 and AGS3 may inhibit heterotrimer formation or actively promote subunit dissociation “releasing” Gβγ. Such mechanisms of G-protein activation dramatically differ from that involving a typical G-protein coupled receptor and might lead to selective activation of Gβγ-regulated effectors. Alternatively, AGS2 and AGS3 may position G-proteins within a signal transduction complex or regulate signal processing by compartmentalization. Increasing evidence indicates that Gα and Gβγ may exist independently of each other in the cell and perhaps AGS2 and AGS3 serve as alternative functional binding partners for the two G-protein subunits (22-24).

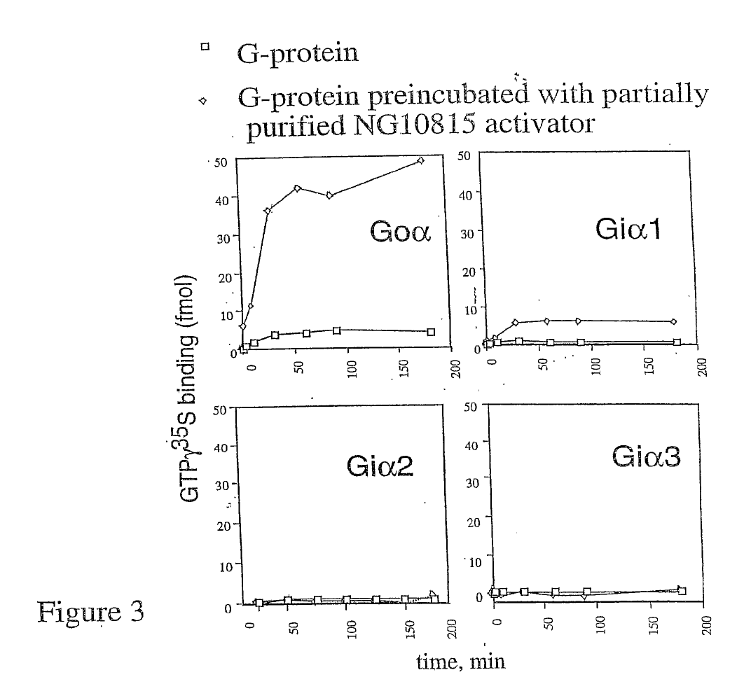

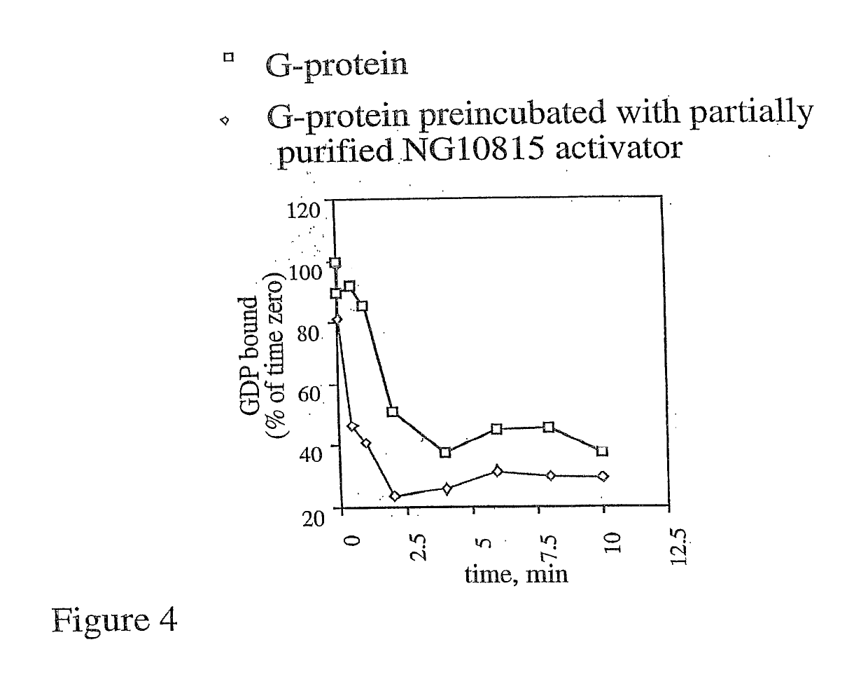

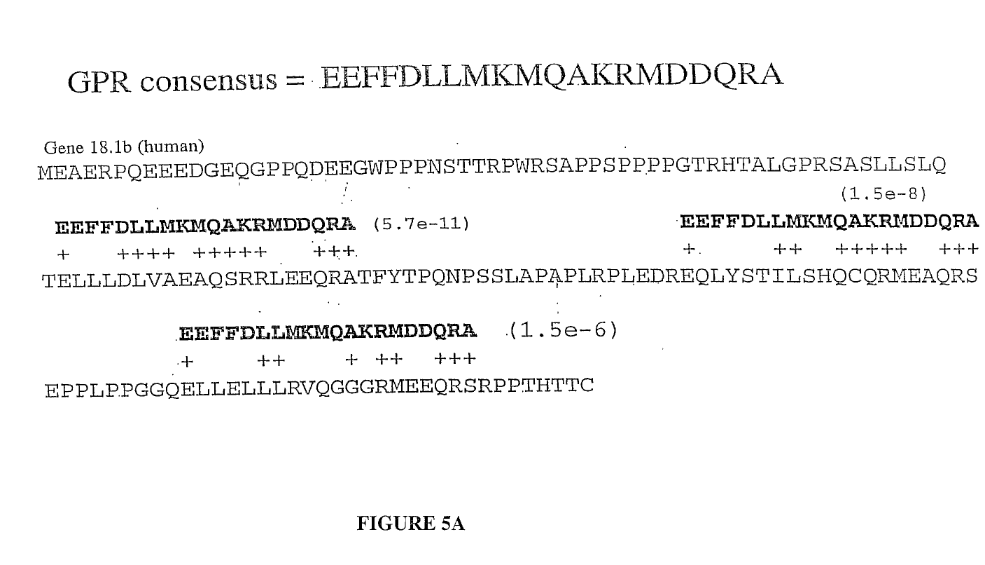

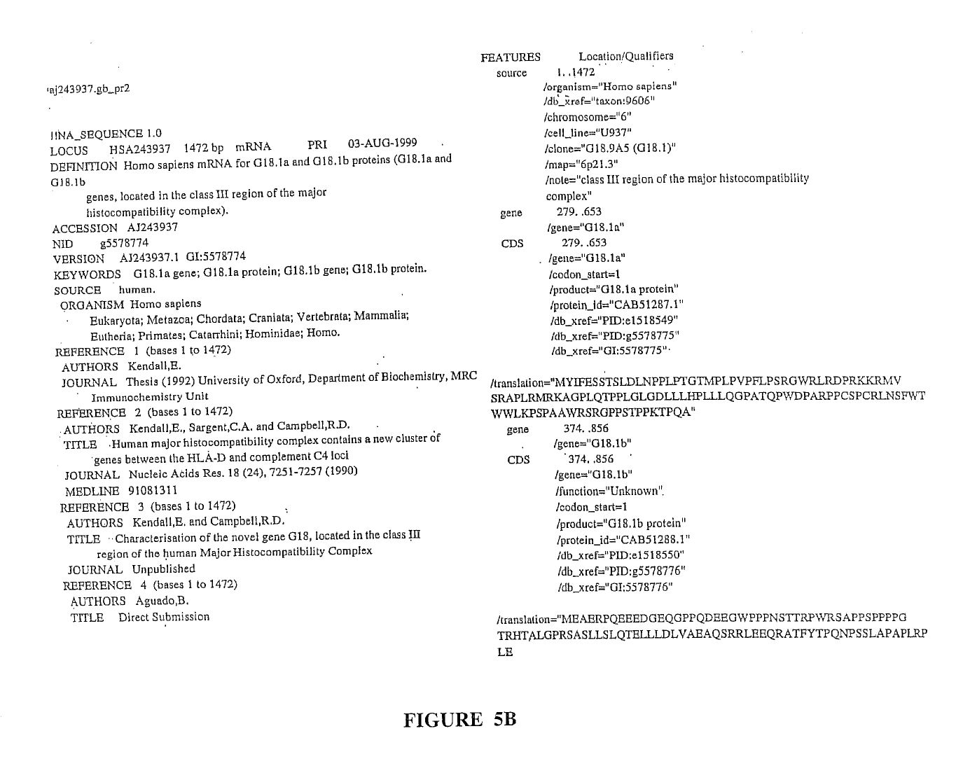

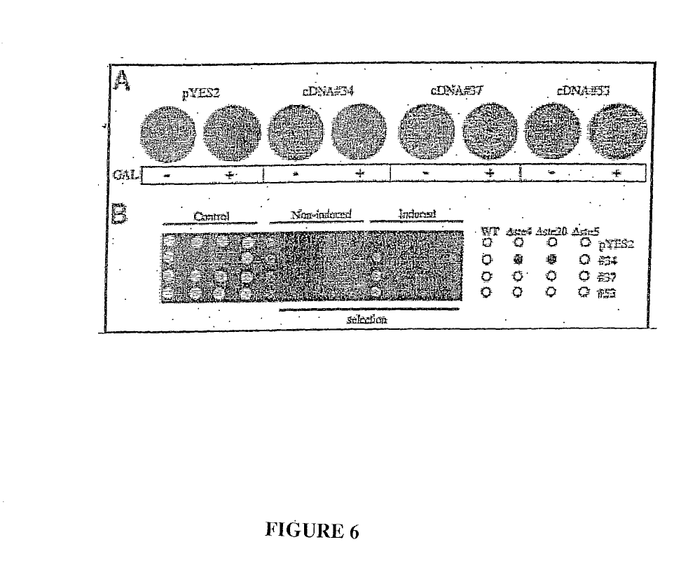

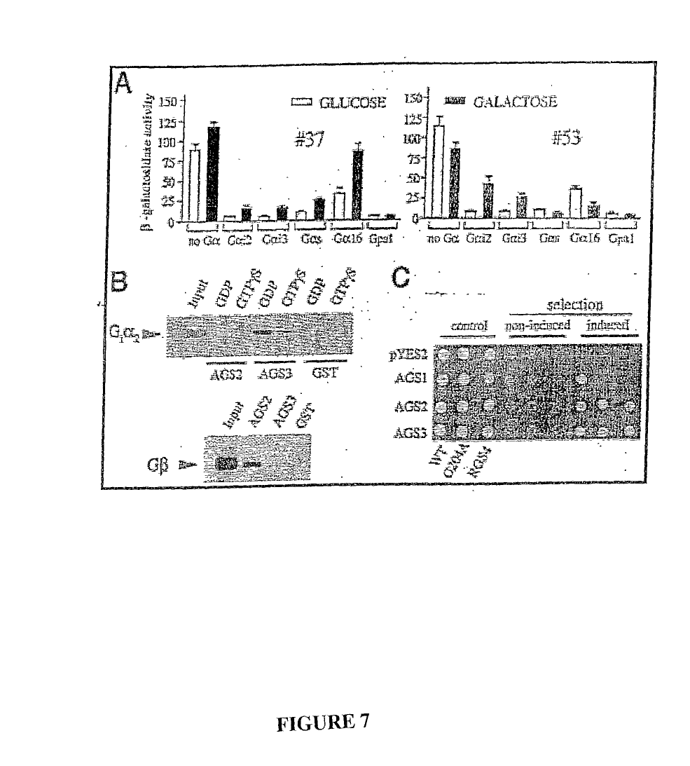

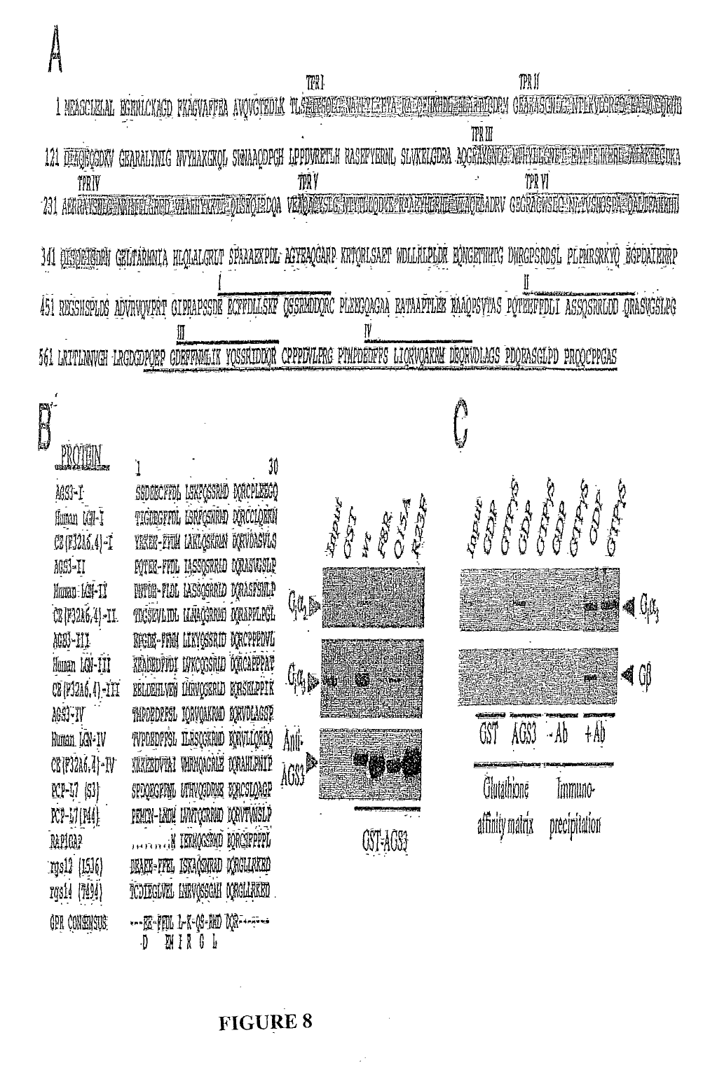

A full length rat AGS3 cDNA (650 amino acids; AF107723) was identified by screening a rat brain cDNA library and subsequent 5′RACE (FIG. 3A). RNA blot analysis identified two AGS3 mRNA species (˜2,300 and ˜4000 nt) that were differentially enriched in heart and brain. The full length AGS3 protein, generated as an amino terminus HIS-tagged protein in Sf9 cells, also interacts with GαGDP as described earlier for the AGS3 subdomain isolated in the yeast screen. Endogenous full length AGS3 and endogenous Giα3 apparently form complexes within the cell as determined in co-immunoprecipitation experiments with crude cell lysates.² Blastp analysis of non-redundant GenBank CDS translations, PDB, SwissProt and PIR indicated that full length AGS3 exhibited homology with the partial mouse cDNA 123316 (96% identity, 97% similarity), the human LGN protein (59% identity, 66% similarity) and a predicted C. elegans protein (PID:g1065449; gene F32A6.4, U40409) (30% identity, 42% similarity). Human LGN was isolated as a truncated carboxyl terminus fragment in a two-hybrid screen using Giα2 as “bait” (26). Analyis of human and mouse EST databases indicated that AGS3 and LGN are not species homologs and that AGS3 and human LGN likely encode two distinct members of a larger protein family defined by conserved structural motifs and functional properties. AGS3, human LGN and the predicted C. elegans protein actually possess two defined structural cassettes that essentially divide the protein in half. The amino terminus half of the AGS3 consists of six tetratricopeptide repeats (TPR repeats), which serve as protein interaction motifs and regulatory domains in various proteins. The TPR repeats in AGS3 may function as a regulatory domain controlling the bioactivity of the carboxyl terminus region or the trafficking of AGS3 and associated proteins within the cell (27-29).

The carboxy terminal half of AGS3 contains four repeated sequences of 18-19 amino acids that exhibit 80-85% homology. The four repeated domains are also found in human LGN and the predicted C. elegans protein. One or two such repeat domains are also found in four proteins that influence the nucleotide binding properties or GTPase activity of G-proteins (FIG. 3B). PcpL7 (PQ0109) (Pcp2) was recently isolated in a yeast two-hybrid screen using Goα as “bait” and may act as a guanine nucleotide exchange factor (30). RAP1GAP (P47736) is a GTPase activating protein for the small G-protein RAP-1A and was also recently identified in a yeast two-hybrid screen using Goα as “bait” (31). RGS12 (AF035151) and RGS14 (O087737) are members of the family of “Regulators of G-protein Signalling”. Analysis of repeated domains I-IV in AGS3, human LGN, the predicted C. elegans protein and the motifs in PcpL7, RGS12, RGS14 and RAP1GAP indicated a consensus sequence of E/DEXF/LFD/EL/ML/IXR/KXQS/GXRM/LDDQR with the X14 position likely hydrophilic. The presence of such a repeated structural motif infers functionality and this consensus sequence is referred to as a G-protein regulatory (GPR) motif. The GPR motif may be a signature for proteins that interact with G-proteins and regulate subunit interactions, nucleotide exchange, GTP hydrolysis and/or the interaction of the G-protein with other entities involved in signal transduction.

Further analysis indicated that each GPR motif can exist as an amphipathic helix. The AGS3 cDNA isolated in the yeast screen actually contained one complete GPR motif. Introduction of mutations into this GPR motif that disrupt the predicted amphipathic helix eliminated interaction of AGS3 with Giα2 and Giα3 in crude cell extracts (FIG. 3B) and the same fusion proteins were inactive in the yeast assay system consistent with the hypothesis stated above regarding functionality of this repeat motif. As observed for the interaction between AGS3 and purified recombinant Giα2 (FIG. 2B), AGS3 preferentially interacted with GαGDP versus GTPγS in the crude cell lysate (FIG. 3C). Despite the presence of GDP, which would stabilize the G-protein heterotrimer, the AGS3-GαGDP complex from the mammalian cell lysate did not contain Gβγ. In contrast, Gβγ subunits were readily detected when GαGDP was isolated from the same cell extract by immunoprecipitation with a Gα subunit antibody (FIG. 3C). These data support the hypothesis that AGS3 activates G-protein signalling by influencing subunit interactions. Alternatively, it is possible that AGS3 is selectively interacting with a population of Gα in the cell that exists independent of Gβγ and subserves unexpected functional roles.

AGS proteins are indicative of a growing number of accessory proteins that influence signal propagation by heterotrimeric G-protein systems (3, 4 and references therein,21, 30, 32). Such entities may influence the population of activated G-protein/effector within the cell independent of external stimuli or provide a cell-specific mechanism for signal amplification by acting in concert with G-protein coupled receptors. Such proteins also provide a mechanism for signal input to heterotrimeric G-protein signalling systems that is distinct from that initiated by a seven-membrane span hormone receptor. By virtue of their distinct properties, AGS2 and AGS3 may also belong to a larger group of proteins that serve as binding partners for Gα and Gβγ allowing the subunits to subserve functions that do not require initial heterotrimer formation.

### Example II

The G-protein regulatory (GPR) motif in AGS3 was recently identified as a region for protein binding to heterotrimeric G-protein a subunits. To define the properties of this ˜20-amino acid motif, we designed a GPR consensus peptide and determined its influence on the activation state of G-protein and receptor coupling to G-protein. The GPR peptide sequence (28 amino acids) encompassed the consensus sequence defined by the four GPR motifs conserved in the family of AGS3 proteins. The GPR consensus peptide effectively prevented the binding of AGS3 to Giα1, 2 in protein interaction assays, inhibited guanosine 5′-O-(3-thiotriphosphate) binding to Giα, and stabilized the GDP-bound conformation of Giα. The GPR peptide had little effect on nucleotide binding to Goα and brain G-protein indicating selective regulation of Giα. Thus, the GPR peptide functions as a guanine nucleotide dissociation inhibitor for Giα. The GPR consensus peptide also blocked receptor coupling to Giαβγ indicating that although the AGS3-GPR peptide stabilized the GDP-bound conformation of Giα, this conformation of Giα_{GDP} was not recognized by a G-protein coupled receptor. The AGS3-GPR motif presents an opportunity for selective control of Giα- and Gαγ-regulated effector systems, and the GPR motif allows for alternative modes of signal input to G-protein signaling systems.

The G-protein regulatory (GPR)¹ motif or GoLOCO repeat is a ˜20-amino acid domain found in several proteins that interact with and/or regulate G-proteins (33, 34). Such proteins include the activator of G-protein signaling AGS3, the AGS3-related protein PINS in Drosophila melanogaster, two members of the RGS family of proteins, and three proteins (LGN, Pcp2, and Rap1GAP) isolated in yeast two-hybrid screens using Giα or Goα as bait. Rat AGS3 was isolated in a yeast-based functional screen designed to identify receptor-independent activators of heterotrimeric G-protein signaling (1). The AGS3-related protein PINS is required for asymmetric cell division of neuroblasts in D. melanogaster, where it is found complexed with Gi/Go (35, 36), but neither the signal input nor output for this complex is known. Some insight as to how PINS may regulate Gi/Go is provided by studies with AGS3 (33). In the yeast-based system, AGS3 selectively activated Giα2 and Giα3. The action of AGS3 as a G-protein activator in the yeast-based system was independent of nucleotide exchange as it was not antagonized by overexpression of RGS4, and it was still observed following replacement of Giα2 with Giα2-G204A, a mutant that is deficient in making the transition to the GTP-bound state (33, 5). Both of these manipulations effectively prevent receptor-mediated activation of G-protein signaling in the yeast system and block the action of AGS1, which was isolated in the same screen and apparently behaves as a guanine nucleotide exchange factor for heterotrimeric G-proteins (5, 7)). These data indicate that the interaction of AGS3 with G-protein influences a unique control mechanism within the activation/deactivation cycle of heterotrimeric G-proteins.

AGS3 exists as a 650-amino acid protein enriched in brain and a 166-amino acid protein (AGS3-SHORT) enriched in heart (33). The 650-amino acid protein consists of two functional domains defined by a series of seven amino-terminal tetratrico peptide repeats (TPR) and four carboxyl-terminal GPR motifs. Site-directed mutagenesis, protein interaction studies, and subcellular localization experiments indicated that the GPR motifs of AGS3 were likely responsible for binding G-protein, whereas the TPR domain is a site for binding of regulatory proteins (33, 35, 36). AGS3 preferentially binds to Gα in the presence of GDP (33). AGS3-GPR effectively competed with Gβγ subunits for binding to Gtα and inhibited guanosine 5′-O-(3-thiotriphosphate) (GTPγS) binding to Giα1. Such an activity has significance in a number of aspects of G-protein-mediated signaling events and presents a novel opportunity to control the basal activity of G-protein signaling, as well as influence receptor-mediated activation of G-protein. These observations also raise many interesting questions relative to basic aspects of G-protein structure/function and alternative modes of regulation and functional roles for G-protein signaling systems in the cell. To address these issues, a series of peptides were generated based upon the consensus GPR motif in AGS3 and their effects on the nucleotide binding properties of Giα were evaluated. A 28-amino acid GPR peptide effectively blocked the interaction of AGS3 with Giα and inhibited GTPγS binding to Giα by a mechanism that involved stabilization of the GDP-bound conformation of Giα. The GPR consensus peptide also blocked receptor coupling to Giαβγ indicating that although the AGS3-GPR peptide stabilized the GDP-bound conformation of Giα, this conformation of Giα_{GDP} was not recognized by a G-protein-coupled receptor.

Materials—³⁵S-GTPγS (1250 Ci/mmol), ³H-GDP (29.6 Ci/mmol), and ³H-5-hydroxy tryptamine (HT) (21.8 Ci/mmol) were purchased from PerkinElmer Life Sciences. Peptides were synthesized and purified by Bio-Synthesis, Inc. (Lewisville, Tex.), and peptide mass was verified by matrix-assisted laser desorption ionization mass spectrometry. GDP, GTPγS, and 5-HT were obtained from Sigma. Acrylamide, bisacrylamide, protein assay kits, and sodium dodecyl sulfate were purchased from Bio-Rad. Ecoscint A was purchased from National Diagnostics (Manville, N.J.). CytoScint was purchased from ICN Biomedicals (Costa Mesa, Calif.). Thesit (polyoxyethylene-9-lauryl ether) was obtained from Roche Molecular Biochemicals. Polyvinylidene difluoride membranes were obtained from Pall Gelman Sciences (Ann Arbor, Mich.). Nitrocellulose BA85 filters were purchased from Schleicher & Schuell (Keene, N.H.). Whatman GF/C FP200 filters were purchased from Brandel Inc.(Gaithersburg, Md.). Purified bovine brain G-protein was kindly provided by Dr. John Hildebrandt (Department of Pharmacology, Medical University of South Carolina) (37). All other materials were obtained as described elsewhere (33, 37).

Protein Interaction Assays—The GPR domain of AGS3 (Pro⁴⁶³-Ser⁶⁵⁰) containing the four GPR motifs was generated as a glutathione S-transferase fusion protein by polymerase chain reaction using the full-length cDNA of AGS3 as a template. The AGS3-Pro⁴⁶³-Ser⁶⁵⁰ segment was also cloned into the pQE-30 vector (Qiagen, Valencia, Calif.) to generate an amino-terminal His-tagged protein. His-tagged AGS3 was expressed in and purified from bacteria using a nickel affinity matrix (ProBond™ resin; Invitrogen, Carlsbad, Calif.). The His-tagged AGS3 was eluted from the matrix with imidazole and desalted by centrifugation as with the GST fusion protein (33). The interaction of GST-AGS3-GPR and HIS-tagged AGS3-GPR with G-proteins was assessed by protein interaction experiments using purified G-protein as described previously (33). Giα1-3 and Goα were purified from Sf9 insect cells infected with recombinant virus as described (38). All purified G-proteins used in these studies were isolated in the GDP-bound form, and G-protein interaction assays contained 10 μM GDP.

A separate series of protein interaction experiments were designed to determine whether the Giα complexed with AGS3 contained bound GDP. Giα1 (100 nM) was loaded with ³H-GDP (0.5 μM; 2.0×10⁴ dpm/pmol) by incubation for 20 min at 24° C. in binding buffer (50 mM Hepes-HCl, pH 7.5, 1 mM EDTA, 1 mM dithiothreitol, 50 μM adenosine triphosphate, and 10 μg/ml bovine serum albumin). The ³H-GDP-loaded Giα 1 was incubated with 300 nM GST or GST-AGS3-GPR in the presence and absence of 10 μM GPR peptide and processed as described (33). The washed resin containing bound proteins was transferred to vials for measurement of ³H-GDP by liquid scintillation spectroscopy.

GTPγS Binding Assays—GTPγS binding assays were generally conducted as described (39). G-proteins (100 nM) were preincubated for 20 min at 24° C. in the presence and absence of GPR peptides. Binding assays (duplicate determinations) were initiated by addition of 0.5 μM GTPγS (4.0×10⁴ dpm/pmol), and incubations (total volume=50 μl) were continued for 30 min at 24° C. Both preincubations and GTPγS binding assays were conducted in binding buffer containing 2 mM MgCl₂. Reactions were terminated by rapid filtration through nitrocellulose filters with 4×4-ml washes of stop buffer (50 mM Tris-HCl, 5 mM MgCl₂, 1 mM EDTA, pH 7.4, at 4° C.). Radioactivity bound to the filters was determined by liquid scintillation counting. Nonspecific binding was defined by 100 μM GTPμS.

GDP Dissociation Assays—Giα1 (100 nM) was loaded with ³H-GDP (0.5 μM; 2×10⁴ dpm/pmol) by incubation for 20 min at 24° C. in binding buffer without MgCl₂. 45-μl aliquots of the preincubation mixture (˜500,000 dpm) were then added to incubation tubes containing 5 μl of vehicle or peptide, and samples were incubated for 30 min at 24° C. Two sets of tubes were set up for each time point to be analyzed. Each set contained duplicate samples for determination of total binding, nonspecific binding, or binding in the presence of peptide. For each time point, one set of tubes served as an internal time control, whereas the other set received added GTPγS or GDP to initiate dissociation. Data are expressed as % of control, where control represents the level of ³H-GDP binding at each time point in the set of tubes that did not receive added nucleotide to initiate dissociation. The amount of ³H-GDP bound following the 20-min preincubation (˜30,000 dpm) was identical to that observed at the 30-min incubation time point following addition of vehicle or peptide. ³H-GDP dissociation was initiated by addition of GTPγS or GDP in a volume of 5 μl (final concentration, 100 μM). Reactions were terminated at specified time points by rapid filtration through nitrocellulose filters (BA85; Schleicher & Shuell) with 4×4-ml washes of stop buffer. Radioactivity bound to the filters was determined by liquid scintillation counting. Nonspecific binding was defined by 100 μM GDP.

High Affinity Agonist Binding—Sf9 cell membranes expressing 5-HT_{1A} receptors were reconstituted with Gαβγ, and high affinity agonist binding was measured with ³H-5-HT as described previously (38, 40). Membrane aliquots (100 μg of membrane protein, 85 nM receptor) were preincubated for 15 min at 25° C. with G-proteins (2125 nM Gαβγ) with or without GPR peptides in a total volume of 17 μl (reconstitution buffer, 5 mM NaHEPES, 100 mM NaCl, 5 mM MgCl₂, 1 mM EDTA, 500 nM GDP, 0.04% CHAPS, pH 7.5). The reconstitution mixtures were then diluted 10-fold with binding buffer (50 mM Tris-HCl, 5 mM MgCl₂, 0.5 mM EDTA, pH 7.5), and 50 μl were added to binding tubes (total volume=150 μl) containing 2 nM ³H-5-HT. The final concentrations of receptor, G-protein, and peptide in the binding tubes were 2.8 nM, 70.8 nM, and 114 μM, respectively. Nonspecific binding was determined in the presence of 100 μM 5-HT. Binding reactions were incubated at 25° C. for 1.5 h and terminated by filtration over Whatman GF/C FP200 filters using a Brandel cell harvester. The filters were rinsed thrice with 4 ml of ice-cold washing buffer (50 mM Tris-Cl, 5 mM MgCl₂, 0.5 mM EDTA, 0.01% sodium azide, pH 7.5, at 4° C.), placed in 4.5 ml of CytoScint, and counted to constant error in a scintillation counter.

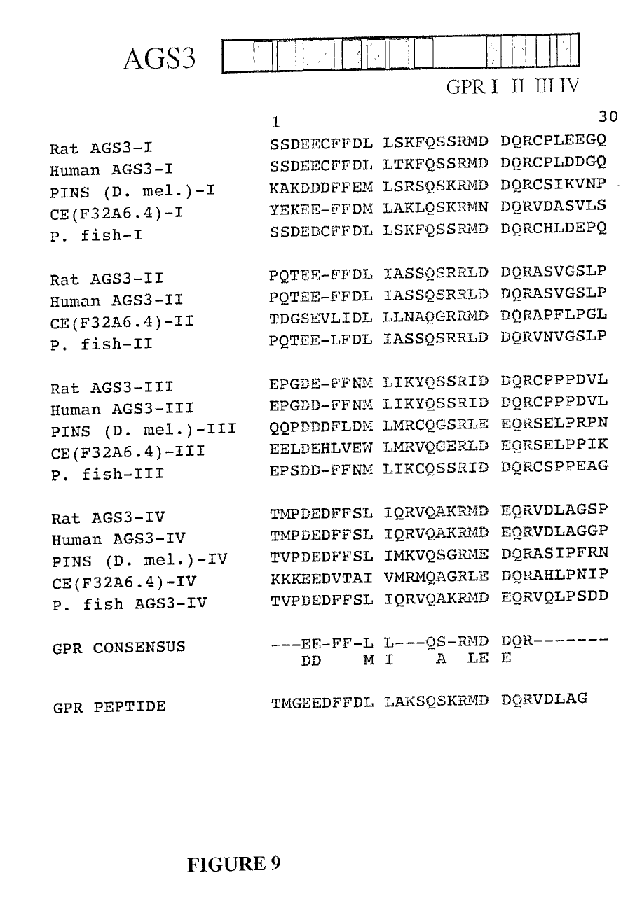

The ˜20-amino acid GPR motif is repeated four times in AGS3-related proteins, with the exception of the three repeats found in the Drosophila protein PINS (FIG. 9) Alignment of the four GPR repeats from five species revealed a GPR consensus sequence (FIG. 9). The GPR consensus sequence is characterized by the upstream negative charge (Glu-Glu) and hydrophobic cluster (Phe-Phe), Leu/Met¹⁰, Leu/Ile¹¹, Gln¹⁵, Ser/Ala¹⁶, Arg¹⁸, Met/Leu¹⁹, and the Asp-Asp-Gln-Arg sequence at the carboxyl end of the motif. Helical wheel and Chou-Fasman analysis indicated that this region is capable of existing as an amphipathic helix. Each of the GPR motifs illustrated in FIG. 9 possess a varying number of Proline residues just after and in some cases before the core consensus sequence, which may exert an important influence within the overall organization of the four GPR motifs. As part of an effort to define the structural basis of the interaction of AGS3 with Giα and the functional consequences of this interaction, whether or not a consensus sequence peptide effectively interacted with Giα was analyzed.

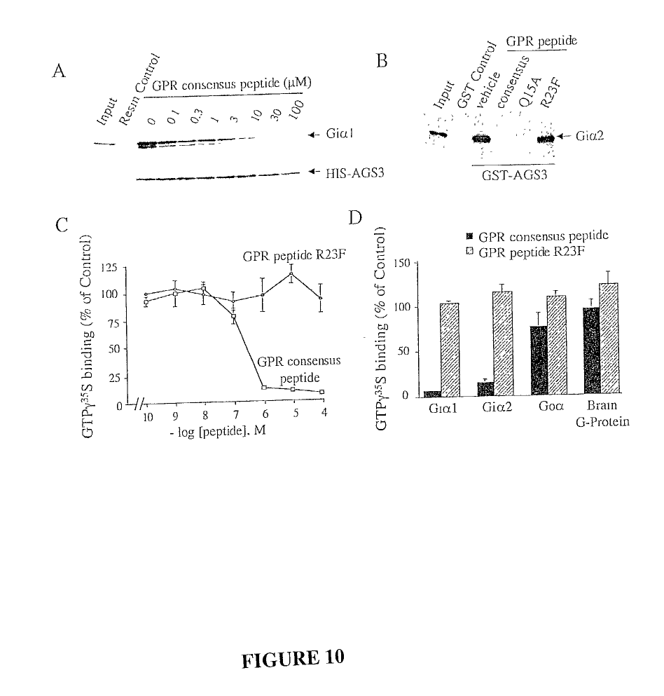

The core GPR consensus sequence was bracketed by additional residues (three amino terminus, five carboxyl terminus) derived from AGS3-GPR-IV, and the carboxyl terminus was amidated (FIG. 9). The 28-amino acid GPR consensus peptide completely blocked the binding of Giα1 or Giα2 to GST-AGS3-GPR with an IC₅₀ of ˜200 nM (FIGS. 10A and B). The GPR consensus peptide also inhibited GTPγS binding to Giα1 and Giα 2 (IC₅₀˜200 nM) (FIGS. 10, C and D) consistent with the preferential binding of AGS3 to Giα in the presence of GDP (33). The inhibitory effect of the GPR consensus peptide on GTPγS binding was selective for Giα as it only minimally affected nucleotide binding to Goα or brain G-protein (FIG. 10D). The activity of the GPR consensus peptide in both the protein interaction assays and GTPγS binding assays was lost upon substitution of Phe for the highly conserved Arg²³ (FIGS. 10, B, C, and D). However, substitution of Ala for the invariant Gln¹⁵ did not alter the activity of the GPR peptide (FIG. 10B). Similar results were obtained when these amino acid substitutions were made in the context of GST-AGS3 fusion protein, which contained the terminal 74 amino acids of AGS3 including part of GPR-III and all of GPR-IV (FIG. 9) (33).

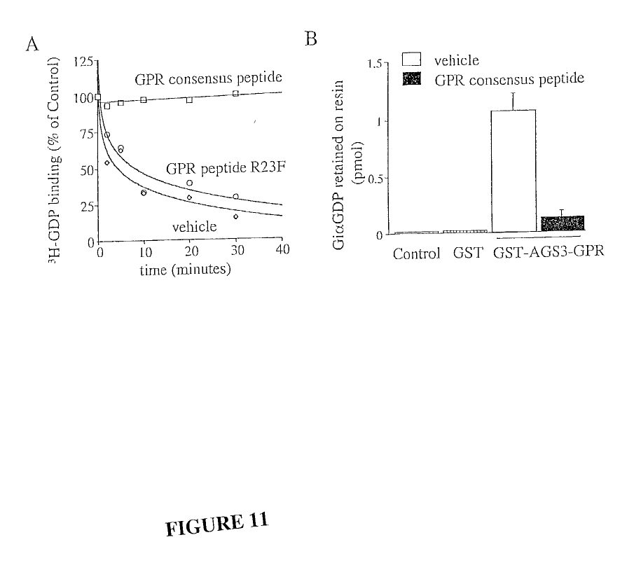

The mechanism by which the GPR consensus peptide inhibited GTPγS binding to Giα2 was addressed and the effect of the GPR motif on receptor coupling to G-protein was determined. The inhibition of GTPγS binding to Giα by the GPR consensus peptide may reflect a reduction in the rate of nucleotide exchange. Indeed, the rate of GDP dissociation was markedly diminished in the presence of the GPR consensus peptide (FIG. 11A). The R23F mutation, which eliminated the effectiveness of the peptide to block interaction of AGS3 with Giα and GTPγS binding to Giα, also did not alter GDP dissociation (FIG. 11A). The inhibition of GDP dissociation by the GPR consensus peptide suggests that the GPR motif is stabilizing the GDP-bound conformation of Giα. To address this issue the interaction of GST-AGS3-GPR with Giα2, which had been preloaded with ³H-GDP was evaluated. Subsequent analysis of the G-protein complexed with AGS3-GPR on the glutathione affinity matrix indicated that the nucleotide binding site of G-protein bound to AGS3 indeed contained GDP (FIG. 11B). Giα_{GDP} binding to AGS3-GPR was blocked by the GPR consensus peptide (FIG. 11B) consistent with the ability of this peptide to inhibit interaction of GST-AGS3-GPR with Gia1/2 (FIGS. 10, A and B).

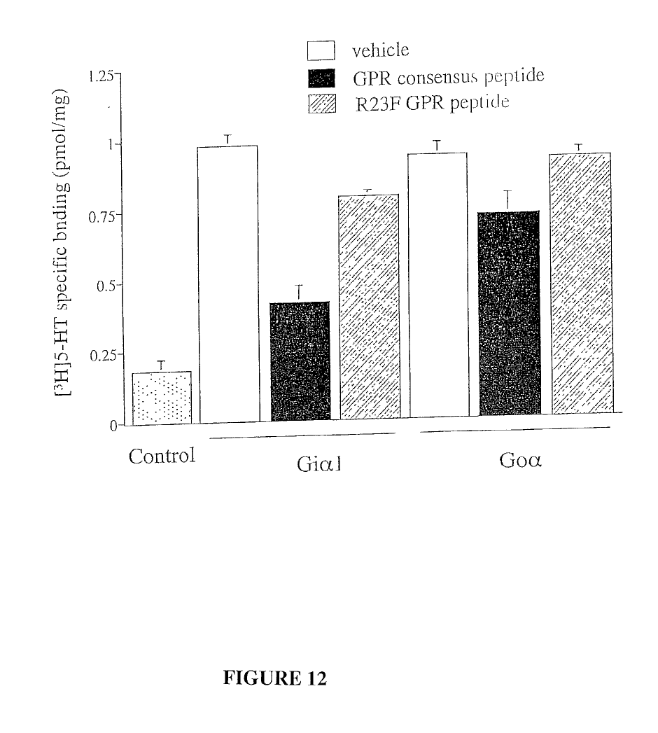

The stabilization of the GDP-bound conformation of Giα by the GPR consensus peptide indicates that the AGS3-GPR motif can influence subunit interactions by interfering with Gβγ binding to Giα.² This apparent effect may account for the results obtained in protein interaction assays using GST-AGS3-GPR and brain lysates, where Gβγ is absent from the AGS3-Giα complex (33). The influence of the GPR motif on subunit interactions would have significant implications for signal processing. First, interaction of the AGS3-GPR motif with Gαβγ would release Gβγ for regulation of downstream signaling events, while stabilizing Gα_{GDP} (33). Such a mode of signal input may be of utility where there is a need for selective regulation of Gβγ-sensitive effectors. The time frame for termination of such a signaling event (i.e. reassociation of Gβγ with Giα_{GDP}) likely differs from that of a more typical signaling event in which there has been an exchange of nucleotide bound to Giα, and signal termination involves GTP hydrolysis along with subunit reassociation. A second implication of stabilization of Gα_{GDP} by a GPR domain is related to receptor G-protein coupling. This issue was addressed experimentally using a membrane assay system where receptor-G-protein coupling is reflected as high affinity binding of agonists. The high affinity binding of agonist observed upon reconstitution of the membrane-bound 5-HT₁ receptor with Giαβ γ was inhibited by addition of the GPR peptide (FIG. 12). This action of the GPR peptide was not observed with the R23F peptide and was selective for Gi versus Go (FIG. 12).

The influence of the single amino acid substitution on the bioactivity of the GPR both within the context of a short peptide and a GST fusion protein containing an additional 74 amino acids of AGS3 sequence strongly suggest a relatively discrete and specific surface interaction with Giα (FIG. 10) (33). Helical wheel projections and 3D models indicated that when the GPR consensus peptide is fixed in an α helical conformation, the Phe⁸, Ala¹², Gln¹⁵, Met¹⁹, and Arg²³ residues are on the same face of the helix. On this face of the helix is a hydrophobic sector defined by Phe⁸, Ala¹², and Met¹⁹ that is bound by polar residues, which may be involved in charge pairing to residues in Giα. As was the case for the R23F substitution, disruption of this hydrophobic sector by substitution of Arg for Phe⁸ also resulted in a loss of activity for the GST-AGS3-GPR fusion protein in GTPγS binding and protein interaction assays (33).² Thus, either extension (R23F substitution) or shortening (F8R) of the hydrophobic sector on this face of the helix resulted in a loss of bioactivity for the GPR motif. In contrast, strengthening of this hydrophobic sector by substitution of Ala for Gln¹⁵ did not alter the activity of the GPR peptide. These data indicate an important role for a spatially constrained hydrophobic stretch of ˜16.6 Å that is key for peptide interaction with Giα.

The inability of receptor to productively couple to Gα_{GDP}-GPR is of interest. The Gα_{GDP} conformation stabilized by the GPR peptide may differ from that stabilized by Gβγ in such a manner that the receptor cannot recognize Gα. Indeed, the orientations of the amino and carboxyl domains of Giα1, which are important interaction sites with receptor, are quite different in the Giα_{GDP} and Giα_{GDP}βγ structures (41-43). In addition to such differences in the structural orientation of Giα domains interacting with receptor, it is likely that receptor contact points on Gβγ also play a role in receptor-mediated activation of guanine nucleotide exchange (2, 44-47). Alternatively, the receptor may indeed interact with the Gα_{GDP}-GPR complex, but this interaction stabilizes a receptor conformation with low affinity for agonist (48). Ultimately, one may think of the Gα_{GDP}-GPR complex as a type of dimeric G-protein.

Although the GPR motif is present in several proteins that interact with Gα and/or regulate nucleotide binding/hydrolysis (33, 34) these proteins have different and often opposing effects on the activation state of G-protein (49, 30).^{2,5} Pcp2, which contains two GPR motifs based upon this consensus sequence, actually appears to increase the dissociation of GDP from Goα (30). Thus, there are either subtle differences in this motif or other residues outside of this motif that play a key role in the specific functional output gendered by interaction of the GPR motif with Gα. Of note is the selective effects of the AGS3-GPR peptide for Giα versus Goα in both nucleotide binding assays and the analysis of receptor coupling to G-proteins. Further dissection of the structural basis for this selectivity will provide clues as to the site of interaction of the GPR peptide with Giα and the mechanism by which it stabilizes the GDP-bound conformation. One prominent area of sequence divergence between Goα and Giα encompasses switch IV, a region implicated in the formation of Gi1α_{GDP} multimers (41).

The role of AGS3 as a GDI is an unexpected concept for heterotrimeric G-proteins, although such proteins serve similar regulator roles for Ras-related G-proteins. Proteins containing the AGS3-GPR motif may promote dissociation of Gα and Gβγ in the absence of nucleotide exchange and present an opportunity for selective control of Giα- and Gβγ-regulated effector systems. GPR-containing proteins likely play a role in regulating basal activity of G-protein signaling systems in the cell and provide alternative modes of signal input to G-protein signaling systems that may either augment, complement, or antagonize G-protein activation by GPCRs.

Throughout this application, various publications are referenced. The disclosures of these publications in their entireties are hereby incorporated by reference into this application in order to more fully describe the state of the art to which this invention pertains.

### References

1. Wu, G., Krupnick, J. G., Benovic, J. L., and Lanier, S. M. (1997) J. Biol. Chem. 272, 17836-17842.

2. Wu, G., Hildebrandt, J., Benovic, J. L., and Lanier, S. M. (1998) J. Biol. Chem. 273, 7197-7200

3. Sato, M., Kataoka, R., Dingus, J., Wilcox, M., Hildebrandt, J., and Lanier, S. M. (1995) J. Biol. Chem. 270, 15269-15276

4. Sato, M., Ribas, C., Hildebrandt, J. D., and Lanier, S. M. (1996) J. Biol. Chem. 271, 30052-30060

5. Cismowski, M., Takesono, A., Ma, C., Lizano, J. S., Xie, S., Fuernkranz, H., Lanier, S. M., and Duzic, E. (1999) Nature Biotechnol. 17, 878-883

6. Schultz, J., Ferguson, B., and Sprague, G. F. Jr. (1995) Curr. Op. Gen. & Devel. 5, 31-37

7. Cismowski, M., Ma, C., Ribas, C., Xie, X., Spruyt, M., Lizano, J. S, Lanier, S. M., and Duzic, E. (2000) J. Biol. Chem. 275, 23421-23424.

8. Lanier, S., M., Downing, S., Duzic, E. and Homcy, C. J. (1991) J Biol Chem 266:10470-10478

9. Schultz, J., Milpetz, F., Bork, P., and Ponting, C. P. (1998) Proc. Natl. Acad. Sci. USA 95, 5857-5864

10. Bailey, T. L. and Gribskov, M. J. (1997) J. Comput. Biol. 4, 45-59

11. Fischer, D. and Eisenberg, D. (1997) Proc. Natl. Acad. Sci. USA. 94, 11929-11934

12. Holbrook, S. R. et al (1993) Biotechniques 14, 984-989

13. Klein, C., Paul, J. I., Sauve, K., Schmidt, M. M., Arcangeli, L., Ransom, J., Trueheart, J., Manfredi, J. P., Broach, J. R. and Murphy, A. J. (1998) Nature Biotech. 16, 1334-1337

14. King, S. M., et al (1996) J. Biol. Chem. 271, 32281-32287

15. Harrison, A., Olds-Clarke, P., and King, S. M. (1998) J. Cell Biol. 140, 1137-1147

16. Tai, A. W., Chuang, J. Z., and Sung, C. H. (1998) J. Biol. Chem. 273, 19639-19649

17. Nagano, F. Orita, S., Sasaki, T., Naito, A., Sakaguchi, G., Maeda, M., Watanabe, T., Kominami, E., Uchiyama, Y., and Takai, Y. (1998) J. Biol Chem. 273, 30065-30068

18. Campbell, K. S., Cooper, S., Dessing, M., Yates, S., and Buder, A. (1998) J Immunol 161, 1728-1737

19. Tai, A. W., Chuang, J-Z., Bode, C., Wolfrum, U. and Sung, C-H. (1999) Cell 97, 877-887

20. Lagriffoul, A. Charpentier, N., Carrette, J., Tougard, C., Bockaert, J. and Homburger, V. (1996) J. Biol. Chem. 271, 31508-31516

21. Roychowdhury, S and Rasenick, M. M. (1997) J. Biol. Chem. 272, 31576-31581

22. Lin, H. C. Duncan, J. A., Kozasa, T. and Gilman, A. G. (1998) Proc. Natl. Acad. Sci. USA 95, 5057-5060

23. Denker, S. P., Mccaffery, J. M., Palade, G. E., Insel, P. A., and Farquhar, M. G. (1996) J. Cell Biol. 133, 1027-1040

24. Jamora, C., Yamanouye, N., Van Lint, J., Laudenslager, J., Vandenheede, J. R., Faulkner, D. J. and Malhotra, V. (1999) Cell 98, 59-68

25. Watson, N., Linder, M. E., Druey, K. M., Kehrl, J. H., and Blumer, K. J. (1996) Nature 383, 172-175

26. Mochizuki, N., Cho, G., Wen, B., and Insel, P. A. (1996) Gene 181, 39-43

27. Carrello, A., Ingley, E., Minchin, R. F., Tsai, S. and Ratajczak, T. (1999) J. Biol Chem 274, 2682-9, 1999

28. Ramarao, N. K. and Cohen, J. B. (1998) Proc. Natl. Acad. Sci. USA 95, 4007-4012

29. Das, K., Cohen, P. T. W., and Barford, D. (1998) EMBO J. 17, 1192-1199

30. Luo, Y. and Denker, B. M. (1999) J. Biol Chem. 274, 10685-10688

31. Jordan, J. D., Carey, K. D., Stork, P. J. S. and Iyengar R. (1999) J. Biol Chem. 274, 21507-21510

32. Lanier, S. M. (1999) Handbook Of Experimental Pharmacology Series, Springer-Verlag. Series Editors—Born, Cuatrecasas, Ganten, Herkan and Melmon. Volume editors—Kenakin and Angus. Springer-Verlag.

33. Takesono, A., Cismowski, M. J., Ribas, C., Bernard, M., Chung, P., Hazard, S., III, Duzic, E., and Lanier, S. M. (1999) J. Biol. Chem. 274, 33202-33205.

34. Siderovski, D. P., Diverse-Pierlussi, M. A., and De Vries, L. (1999) Trends Biochem. Sci. 24, 340-341.

35. Yu, F. W., Morin, X., Cai, Y., Yang, X. H., and Chia, W. (2000) Cell 100, 399-409.

36. Schaefer, M., Schevchenko, A., Schevchenko, A., and Knoblich, J. A. (2000) Curr. Biol. 10, 353-362.

37. Dingus, J., Wilcox, M. D., Kohnken, R., and Hildebrandt, J. D. (1994) Methods Enzymol. 237, 457-471.

38. Clawges, H. M., Depree, K. M., Parker, E. M., and Graber, S. G. (1997) Biochemistry 36, 12930-12938.

39. Ross, M., and Higashijima, T. (1994) Methods Enzymol. 237, 26-37.

40. Bae, H., Anderson, K., Flood, L. A., Skiba, N. P., Hamm, H. E., and Graber, S. G. (1997) J. Biol. Chem. 272, 32071-32077.

41. Mixon, M. B., Lee, E., Coleman, D. E., Berghuis, A. M., Gilman, A. G., and Sprang, S. R. (1995) Science 270, 954-960.

42. Wall, M. A., Coleman, D. E., Lee, E., Iniguezlluhi, J. A., Posner, B. A., Gilman, A. G., and Sprang, S. R. (1995) Cell 83, 1047-1058.

43. Sprang, S. R. (1997) Ann. Rev. Biochem. 66, 639-678.

44. Kleuss, C., Scherubl, H., Hescheler, J., Schultz, G., and Wittig, B. (1992) Science 358, 424-426

45. Taylor, J. M., Jacob-Mosier, G. G., Lawton, R. G., Remmers, A. E., and Neubig, R. R. (1994) J. Biol. Chem. 269, 27618-27624.

46. Hildebrandt, J. D. (1997) Biochem. Pharmacol. 54, 325-339.

47. Richardson, M., and Robishaw, J. D. (1999) J. Biol. Chem. 274, 13525-13533.

48. Nanoff, C., and Freissmuth, M. (1997) Physiol. Res. 46, 79-87.

49. Berman, D. M., and Gilman, A. G. (1998) J. Biol. Chem. 273, 1269-1272.

What is claimed is:

1. A polypeptide of less than 600 amino acids comprising the sequence set forth as SEQ ID NO:1.

2. A polypeptide consisting of the sequence set forth as SEQ ID NO:1.

3. An isolated nucleic acid sequence encoding the polypeptide of claim 1.

4. A ligand that specifically binds to the polypeptide of claim 2.

5. The ligand of claim 4, wherein the ligand is a purified antibody which specifically binds to SEQ ID NO: 1.

6. A method of identifying a polypeptide as a putative receptor-independent activator of heterotrimeric G-protein signaling pathways comprising detecting SEQ ID NO:1 in a polypeptide, wherein the presence of SEQ ID NO:1 in the polypeptide indicates the polypeptide is a putative receptor-independent activator of heterotrimeric G-protein signaling pathways.

7. The method of claim 6, wherein detecting SEQ ID NO:1 comprises comparing the sequence of the polypeptide with SEQ ID NO:1.

8. A method of identifying compounds that interact with the polypeptide of claim 1 comprising:
a) contacting the polypeptide of claim 1 with the compound;
b) determining whether the compound interacts with the polypeptide of claim 1 and;
c) determining whether the compound interacts with SEQ ID NO:1 of the polypeptide of claim 1.

9. A method of identifying a compound that inhibits the interaction of the polypeptide of claim 1 and a G-protein subunit comprising:
a) contacting the polypeptide of claim 1 with a G-protein subunit;
b) administering the compound;
c) determining whether the compound inhibits the interaction of the polypeptide of claim 1 and the G-protein subunit and;
d) determining whether the compound interacts with SEQ ID NO:1 of the polypeptide of claim 1, thereby identifying a compound that inhibits the interaction of the polypeptide of claim 1 and a G-protein subunit.

10. A method of identifying a compound that inhibits the interaction of the polypeptide of claim 1 and a G-protein comprising:
a) expressing the polypeptide of claim 1 in a cell having a receptor independent G-protein signaling pathway;
b) detecting receptor independent activation of G-protein signaling in the cell;
c) administering a compound to the cell of b);
d) detecting inhibition of receptor independent activation of G-protein signaling and;
e) determining whether the compound interacts with SEQ ID NO:1 of the polypeptide of claim 1, thereby identifying a compound that inhibits the interaction of the polypeptide of claim 1 and a G-protein.

11. A method of inhibiting the interaction of the polypeptide of claim 1 and a G-protein comprising administering to a subject a ligand that specifically binds to the amino acids in the sequence set forth as SEQ ID NO: 1.

12. A method of screening for a mimetic or analog of SEQ ID NO: 1 or a fragment thereof, comprising:
a) contacting a model system known to express an activity of SEQ ID NO: 1 with a putative mimetic or analog
b) detecting the presence or absence of the known activity of SEQ ID NO: 1 in the presence of the putative mimetic or analog; and
c) correlating the presence or absence of the activity with the presence of a mimetic of SEQ ID NO: 1.

13. The method of claim 12, wherein the activity of SEQ ID NO: 1 is a stimulatory activity.

14. The method of claim 13, wherein the stimulatory activity is activation of G-protein mediated signal transduction.

15. The method of claim 12, wherein the activity of SEQ ID NO: 1 is an inhibitory activity.

16. The method of claim 15, wherein the inhibitory activity is inhibition of G-protein mediated signal transduction.

17. A method of screening for an antagonist of SEQ ID NO: 1 comprising:
a) contacting a model system known to express an activity of a polypeptide comprising a GPR motif with a putative antagonist in the presence of SEQ ID NO: 1 or a fragment thereof;
b) detecting the absence of, or reduction in, the known activity of the polypeptide comprising a GPR motif, in the presence of the putative antagonist;
c) correlating the absence of, or reduction in, the activity with the presence of an antagonist of SEQ ID NO: 1 or a fragment thereof and;
d) confirming that the antagonist interacts with SEQ ID NO: 1 or interferes with the binding of SEQ ID NO: 1 to a G-protein.

18. The method of claim 15, wherein the activity of SEQ ID NO: 1 is a stimulatory activity.

19. The method of claim 18, wherein the stimulatory activity is activation of G-protein mediated signal transduction.

20. The method of claim 15, wherein the activity of SEQ ID NO: 1 is an inhibitory activity.

21. The method of claim 13, wherein the inhibitory activity is inhibition of G-protein mediated signal transduction.

22. A method of inhibiting G protein mediated signal transduction comprising administering to a subject a GPR motif or mimetic of a GPR motif.


SEQUENCE LISTING (3 sequences)

SEQ ID NO: 1 - protein, 20 aa, Artificial Sequence
  Description of Artificial Sequence/ Note = synthetic construct
XXXXXXXXXXXQXXXXXXQR

SEQ ID NO: 2 - DNA, 40 bp, Artificial Sequence
  Description of Artificial Sequence/Note = synthetic construct
ataaggctgaagaaatcctcatcaggcatggtagggccac

SEQ ID NO: 3 - protein, 25 aa, Artificial Sequence
  Description of Artificial Sequence/Note = synthetic construct
VDLAGSPEQEASGLPDPQQQYPPGS